(19) United States
(12) Patent Application Publication
(10) Pub. No.: US 2011/0142897 A1
(43) Pub. Date: Jun. 16, 2011

(54) PROCESS FOR TREATMENT OF KERATINOUS MATERIALS AND KIT FOR FORMULATION OF A COSMETIC PRODUCT

(75) Inventors: Thierry COTTON, Paris (FR); Frederique Labatut, Paris (FR); Maud Rieffel, Pommeuse (FR); Guenaelle Martin, Chatenay Malabry (FR); Florence Levy, Paris (FR)

(73) Assignee: L'OREAL, Paris (FR)

(21) Appl. No.: 12/968,400

(22) Filed: Dec. 15, 2010

Related U.S. Application Data
(60) Provisional application No. 61/294,501, filed on Jan. 13, 2010.

(30) Foreign Application Priority Data
Dec. 16, 2009 (FR) 09 59051

(51) Int. Cl. A61K 9/10; A61Q 19/00

(52) U.S. Cl. 424/401

(57) ABSTRACT

Kit for formulation of a cosmetic product in emulsion form containing a composition A containing oil and a gelling agent which is in dispersed form, composition A containing less than 10% by weight of water, and an aqueous composition B, compositions A and B being packed separately. Compositions A and B are intended to be mixed on the spot to form a product in emulsion form which can be a make-up product, or for removing make-up or cleaning the skin or for care of the skin, mucous membranes, integuments or hair. Process of using product.

### REFERENCE TO PRIOR APPLICATIONS

This application claims priority to U.S. provisional application Ser. No. 61/294,501, filed Jan. 13, 2010; and to French patent application 09 59051, filed Dec. 16, 2009, both incorporated herein by reference.

### FIELD OF THE INVENTION

The present invention relates to kits for formulation of products, preferably cosmetic products, and in particular to a kit for formulation of cosmetic products in emulsion form comprising two compositions, the compositions being intended to be mixed on the spot to form a cosmetic product in emulsion form.

The invention also relates to a method for cosmetic treatment of keratinous materials such as the skin, mucous membranes, and keratinous fibres such as the hair, making use of the invention compositions.

### BACKGROUND OF THE INVENTION

Users increasingly wish to have available a “personalized” composition, suitable for the use that they wish to make of it at the moment when they have it available. Thus, depending on their mood, the seasons, the outside temperature or also skin problems, the user aims to have available a composition suitable for his/her need of the moment, for example to have available a composition which is more or less thick, or with a higher or lower active ingredient concentration or having a particular colour or perfume. In particular, consumers are looking for personalized cosmetic products which they can prepare themselves at home.

Moreover, this type of galenical preparation has the advantage of being able to reduce or eliminate the preservatives which are indispensable for the good preservation of a conventional cosmetic product and of being able to propose a range of fragile hydrophilic or lipophilic active principles which a “fresh” formulation makes it possible to stabilise (because of a specific storage life and storage conditions).

Kits are known which are intended for the on the spot preparation of personalized cosmetic compositions, which comprise several separate compositions, and in particular a first so-called “base” composition in emulsion form and additional separately packed compositions containing cosmetic active ingredients, perfumes or colorants, enabling the consumer to personalise the base composition according to his/her needs.

These systems thus make use of already formed emulsions to which various cosmetic additives are added in several stages.

These products for preparation “at home” do not always display optimal cosmetic qualities equivalent, particularly in terms of texture, to the finished products available on the market.

There is thus the need for systems making it possible easily (without special equipment), and rapidly (in a reduced number of stages) to create products in a complex form such as an emulsion displaying good cosmetic properties, comparable to the conventional ready-for-use cosmetic products available on the market.

### DETAILED DESCRIPTION OF THE PREFERRED EMBODIMENTS

One subject of the invention is thus a process for cosmetic treatment of keratinous materials comprising:
having available a composition A containing at least one oil and at least one gelling agent which is in dispersed form in an organic or aqueous solvent, the composition containing less than 10% by weight of water
having available an aqueous composition B containing water, for example exclusively containing water, and possibly at least one water-soluble organic solvent,
the compositions A and B being packed separately,
mixing the compositions A and B on the spot so as to form an emulsion,
   then applying the mixture to the keratinous materials.

A subject of the invention is also a process for cosmetic treatment of keratinous materials comprising:
having available a composition A containing at least one oil and at least one gelling agent which is in dispersed form in an organic or aqueous solvent, the said composition containing less than 10% by weight of water and having an oil content ranging from 30% to 95% by weight,
having available an aqueous composition B,
the compositions A and B being packed separately,
mixing the compositions A and B on the spot so as to form an emulsion,
   then applying the mixture to the keratinous materials.

The compositions A and B are preferably mixed on the spot in order to form the a cosmetic product under the form of an emulsion.

This process makes it possible to create an emulsion displaying a stable cosmetic texture in a reduced number of stages (in particular in a single stage), with no energy input and by simple manual stirring.

The cosmetic product under the form of an emulsion can in particular be used as a product for the care or making up of keratinous materials, or also as a product for cleaning or make-up removal from keratinous materials.

The on the spot mixing can be obtained by simple mixing of the desired quantities (in general predetermined) of the compositions A and B, these quantities being determined on the basis of the final desired aim.

The appropriate dose of composition A and composition B can be obtained by using single-dose presentation forms such as sachets, bags, tubes, ampoules, prefilled syringes, soft capsules, or hot-moulded plastic tubs or boats. An appropriate dose can also be obtained from a multi-dose presentation using a system dispensing a predefined dose. Such a system can be a pump bottle, an aerosol, a pipette or a graduated syringe or a dropper. In particular, the compositions A and B can be packed in the form of a sachet, a bag or a pot.

The final cosmetic product is obtained by on the spot mixing of appropriate quantities of composition A and composition B.

The said cosmetic product is preferably obtained by adding composition A to composition B with mixing. The final product is an emulsion, preferably an oil in water emulsion.

According to an advantageous implementation mode, the composition A represents from 2 to 70% by weight, preferably from 10 to 60% by weight, still better from 20 to 50% by weight relative to the total weight of the final cosmetic product.

A subject of the invention is also a kit for formulation of a cosmetic product in emulsion form comprising:
a composition A containing at least one oil and at least one gelling agent which is in dispersed form in an organic or aqueous solvent, the said composition containing less than 10% by weight of water and having an oil content ranging from 30% to 95% by weight, and
an aqueous composition B exclusively containing water and possibly at least one water-soluble organic solvent,
the compositions A and B being packed separately.

An other object of the invention is a kit for formulation of a cosmetic product in emulsion form comprising:
a composition A containing at least one oil and at least one gelling agent which is in dispersed form in an organic or aqueous solvent, the said composition containing less than 10% by weight of water, and
an aqueous composition B,
the compositions A and B being packed separately and
the composition A representing from 2 to 70% by weight, preferably from 10 to 60% by weight, still better from 20 to 50% by weight relative to the total weight of the compositions A and B.

According to one implementation mode, the formulation kit according to the invention comprises, apart from the compositions A and B, a means for mixing the compositions A and B such as a spatula and/or a container such as a pot intended to contain the final product in emulsion form formed by the mixing of the compositions A and B.

According to one implementation mode, the kit according to the invention can further contain instructions, in particular on an explanatory note, for preparing the cosmetic product.

The mixing of the compositions A and B can typically be effected with simple manual stirring, without other energy input, in particular without heating, and a smooth and homogeneous emulsion is rapidly formed. Preferably, composition A is added to composition B. The final product in emulsion form can be stored in the cold after preparation, in particular at a temperature ranging from 2 to 10° C., for example in the refrigerator, for a period for example ranging from 1 to 8 weeks.

According to one implementation mode, the consumer uses mineral water available on the market as the aqueous composition B.

The kit and process according to the invention can enable the user to adjust the composition, rheology, colour or active ingredient concentration of the final product on the spot.

Further, in the case of the application of active ingredients sensitive to an external stimulus, their incorporation into products prepared on the spot by simple mixing and stored in the cold for a limited period makes it possible to guarantee their efficacy and/or their stability.

These preparation and storage conditions also make it possible to reduce or eliminate preservatives. Thus according to one implementation mode the compositions A and B and the cosmetic product derived from the mixing of the said compositions contain less than 1% of preservatives, or are even free from preservatives.

Preferred embodiments include:

A process for treatment of a keratinous material comprising mixing compositions A and B to form an emulsion and then applying the emulsion to the keratinous material, wherein
composition A comprises at least one oil and at least one gelling agent which is in dispersed form in an organic or aqueous solvent, composition A comprising less than 10% by weight of water;
composition B comprising water and optionally at least one water-soluble organic solvent,
   compositions A and B being separate from one another prior to mixing, optionally being separately packed in a single kit prior to mixing.

A process as above, wherein composition A comprises less than 10% by weight of water and has an oil content ranging from 30% to 95% by weight, and composition B is an aqueous composition.

A process as above, wherein compositions A and B are separately packed in a single kit prior to mixing.

Still other subjects will appear in the detailed description which follows.

### I/ Composition A

Composition A contains less than 10% by weight of water, preferably less than 5% of water, better less than 3% by weight of water, and still better less than 1% by weight of water.

Advantageously, composition A is anhydrous and contains no added water, other than the water contributed via one or more starting materials constituting the said composition.

### Oil

Composition A contains at least one oil (fatty substance liquid at ambient temperature (20-25° C.)) which constitutes in whole or in part the fatty phase of the composition.

The oil can be selected from volatile or non-volatile oils, of plant, mineral or synthetic origin, and mixtures thereof. These oils are physiologically acceptable.

As oils usable in the composition of the invention, for example the following can be cited:
hydrocarbon oils of animal origin, such as perhydrosqualene;
hydrocarbon oils of plant origin, such as the liquid triglycerides of fatty acids containing from 4 to 10 carbon atoms such as the triglycerides of heptanoic or octanoic acids or else, for example, sunflower, maize, soya, pumpkin seed, grape seed, sesame, hazel nut, apricot, macadamia, arara, coriander, castor or avocado oils, the triglycerides of caprylic/capric acids such as those marketed by Stearineries Dubois or those marketed under the names Miglyol 810, 812 and 818 by Dynamit Nobel or jojoba oil and shea butter oil;
synthetic esters and ethers, in particular of fatty acids:
   the fatty esters can for example be selected from those obtained from a linear or branched chain, saturated or unsaturated alcohol having from 1 to 24 carbon atoms and from a linear or branched chain fatty acid having from 3 to 24 carbon atoms. As fatty esters, 2-ethylhexyl caprate/caprylate (or octyl caprate/caprylate), ethyl laurate, butyl laurate, hexyl laurate, isohexyl laurate, isopropyl laurate, methyl myristate, ethyl myristate, butyl myristate, isobutyl myristate, isopropyl myristate, 2-octyl-dodecyl myristate, 2-ethylhexyl monococoate (or octyl monococoate), methyl palmitate, ethyl palmitate, isopropyl palmitate, isobutyl palmitate, 2-ethylhexyl palmitate (or octyl palmitate), butyl stearate, isopropyl stearate, isobutyl stearate, 2-ethylhexyl stearate (or octyl stearate), isopropyl isostearate, isocetyl stearate, isostearyl isostearate, 2-ethylhexyl pelargonate (or octyl pelargonate), 2-ethylhexyl hydroxy-stearate (or octyl hydroxystearate), decyl oleate, diisopropyl adipate, di-2-ethylhexyl adipate (or dioctyl adipate), diisocetyl adipate, 2-ethylhexyl succinate (or octyl succinate), diisopropyl sebacate, 2-ethylhexyl malate (or octyl malate), pentaerythritol caprate/caprylate, 2-ethylhexyl hexanoate (or octyl hexanoate), octyldodecyl octanoate, isodecyl neopentanoate, isostearyl neopentanoate, octyldodecyl neopentanoate, isononyl isononanoate, isotridecyl isononanoate, cetearyl isononanoate, isodecyl isononanoate, isotridecyl isononanoate, lauryl lactate, myristyl lactate, cetyl lactate, myristyl propionate, 2-ethylhexyl 2-ethylhexanoate (or octyl 2-ethylhexanoate), 2-ethylhexyl octanoate (or octyl octanoate), cetyl 2-ethylhexanoate, pentaerythritol tetraisostearate, isopropyl lauroyl sarcosinate (Eldew SL 205 from Unipex), dicaprylyl carbonate (Cetiol CC from Cognis), and the benzoates of C12-C15 fatty alcohols (Finsolv TN from FINETEX) can for example be cited.
   As fatty ethers, dicaprylyl ether (Cetiol EO from Cognis) can be cited.
linear or branched hydrocarbons, of mineral or synthetic origin, such as volatile or non-volatile paraffin oils, and derivatives thereof, vaseline, polydecenes, isohexadecane, isododecane, and hydrogenated polybutene such as oil of Parléam®;
linear alkanes, preferably of plant origin, containing from 7 to 19 carbon atoms, in particular from 9 to 17 carbon atoms, and more particularly from 11 to 14 carbon atoms. They can exhibit a flash point lying in the interval varying from 30 to 120° C., and more particularly from 40 to 100° C. and are preferably liquids at ambient temperature (about 25° C.) and at atmospheric pressure (760 mm Hg).

As examples of linear alkanes suitable for the invention, the alkanes described in the patent applications by Cognis WO 2007/068371, or WO2008/155059 (mixtures of distinct alkanes differing by at least one carbon) can be mentioned. These alkanes are obtained from fatty alcohols, themselves obtained from copra oil or palm oil.

As examples of linear alkanes suitable for the invention, n-heptane (C7), n-octane (C8), n-nonane (C9), n-decane (C10), n-undecane (C11), n-dodecane (C12), n-tridecane (C13), n-tetradecane (C14), n-penta-decane (C15), n-hexadecane (C16), n-heptadecane (C17), n-octadecane (C18), n-nonadecane (C19) and mixtures thereof, and in particular the mixture of n-undecane (C11) and n-tridecane (C13) described in Example 1 of the application WO2008/155059 from Cognis can be cited. The n-dodecane (C12) and n-tetradecane (C14) sold by Sasol respectively under the names PARAFOL 12-97 and PARAFOL 14-97, and mixtures thereof can likewise be cited.

The linear alkane can be used alone or in a mixture of at least two distinct alkanes differing from one another by a carbon number of at least 1, and in particular a mixture of at least two distinct linear alkanes containing from 10 to 14 carbon atoms and differing from one another by a carbon number of at least 2, and in particular a mixture of volatile linear C11/C13 alkanes or a mixture of linear C12/C14 alkanes, in particular an n-undecane/n-tridecane mixture (such a mixture can be obtained according to example 1 or example 2 of WO 2008/155059); oils of this type spread easily on the skin and leave a non-greasy and non-sticky feel,
partially hydrocarbon and/or silicone fluorinated oils such as those described in the document JP-A-2-295912;
silicone oils such as volatile or non-volatile poly-methylsiloxanes (PDMS) with a linear or cyclic silicone chain, which are liquid or pasty at ambient temperature, in particular volatile silicone oils, in particular cyclopolydimethylsiloxanes (cyclomethicones) such as cyclohexadimethylsiloxane and cyclopenta-dimethylsiloxane, polydimethylsiloxanes containing alkyl, alkoxy or phenyl groups, within or at the end of the silicone chain, groups having from 2 to 24 carbon atoms, phenylated silicones such as phenyl-tri-methicones, phenyldimethicones, phenyltrimethylsil-oxidiphenylsiloxanes, diphenyldimethicones, diphenyl-methyldiphenyl trisiloxanes, 2-phenylethyltrimethyl siloxysilicates, and polymethyl-phenylsiloxanes, and
mixtures thereof.

Oils selected from plant oils or oils of plant origin can in particular be cited.

In a specific embodiment, the oil is preferably present at a content ranging from 30% to 95% by weight, preferably from 40 to 90% by weight, and better from 40 to 80% by weight relative to the total weight of the composition A.

The fatty phase of the composition can also contain any usual liposoluble or lipo-dispersible additive such as for example other fatty substances such as waxes, pasty compounds, fatty alcohols or fatty acids.

### Gelling Agent

The gelling agent (or thickener) used in composition A of the kit according to the invention is in dispersed form in an organic and/or aqueous solvent. It is thus able to swell and thicken the composition rapidly when it is placed in contact with the water contributed by composition B.

Preferably the gelling agent is selected from polymers which are in the form of an inverse water-in-oil emulsion.

The quantity (as active ingredient) of gelling agent can range from 0.1% to 25% by weight, preferably from 0.5% to 20% by weight and still better from 1% to 15% by weight relative to the total weight of composition A.

The gelling agent is preferably a polymer which can in particular be selected from i) copolymers of α,β-monoethylenically unsaturated carboxylic acids, in particular acrylic or methacrylic acid and ii) copolymers based on 2-acrylamido-2-methylpropane-sulphonic acid and mixtures thereof.

Preferably, the gelling polymers according to the invention can be partially or totally neutralized by an inorganic base (sodium hydroxide, potassium hydroxide or aqueous ammonia) or an organic base such as mono-, di- or triethanolamine, an aminomethylpropanediol, N-methylglucamine, basic amino acids such as arginine and lysine, and mixtures of these compounds. They are generally neutralized. In the present invention, “neutralized” is understood to mean totally or practically totally neutralized, in other words at least 90% neutralized, polymers.

“Copolymers”, as well as copolymers obtained from two sorts of monomer, is also understood to mean those obtained from more than two sorts of monomer such as the terpolymers obtained from three sorts of monomer.

i) Copolymers of α,β-monoethylenically unsaturated carboxylic acids, in particular acrylic or methacrylic acid, are copolymers obtained by copolymerization of one or more monomers (a) selected from α,β-ethyl-enically unsaturated carboxylic acids or esters thereof, with an ethylenically unsaturated monomer (b) containing a hydrophobic group.

The α,β-monoethylenically unsaturated carboxylic acids constituting the monomer (a) can be selected from many acids and in particular from acrylic acid, methacrylic acid, crotonic acid, itaconic acid, maleic acid, and esters thereof. They are preferably acrylic acid or methacrylic acid, and esters thereof.

The monomer (b) containing a hydrophobic group can in particular be selected from ethoxylated (1 to 50 EO) C₁₂-C₃₀ fatty alcohol acrylates, methacrylates or itaconates, such as steareth-20 methacrylate, ethoxylated (25 EO) behenyl methacrylate, ethoxylated (20 EO) monocetyl itaconate, ethoxylated (20 EO) mono-stearyl itaconate, acrylate modified by polyethoxylated (25 EO) C12-C24 alcohols, and from the acrylates or methacrylates of C₁₂-C₃₀ fatty alcohols such as decyl, lauryl, stearyl, behenyl or melissyl acrylates or methacrylates, and mixtures thereof.

As polymers containing at least one ethoxylated C₁₂-C₃₀ fatty alcohol acrylate, methacrylate or itaconate monomer, acrylates/steareth-20 methacrylate copolymer in the form of an aqueous dispersion, such as the product marketed under the name Aculyn 22 by Rohm & Haas (CTFA name: Acrylates/Steareth-30 Methacrylate copolymer), methacrylic acid/ethyl acrylate/ethoxylated (25 EO) behenyl methacrylate terpolymer, such as the product in aqueous emulsion marketed under the name Aculyn 28 by Rohm & Haas, acrylic acid/ethoxylated (20 EO) monocetyl itaconate copolymer, such as the product in 30% aqueous dispersion marketed under the name Structure 3001 by National Starch, acrylic acid/ethoxylated (20 EO) monostearyl itaconate copolymer such as the product in 30% aqueous dispersion marketed under the name Structure 2001 by National Starch, and acrylates/polyethoxylated (25 EO) C12-C24 alcohol-modified acrylate copolymer, such as the latex with 30-32% copolymer, marketed under the name Synthalen W2000 by 3V SA can in particular be cited.

Among these gelling polymers, in particular copolymers containing at least one ethoxylated C₁₂-C₃₀ fatty alcohol acrylate, methacrylate or itaconate monomer, preferably copolymers containing at least one ethoxylated C₁₂-C₃₀ fatty alcohol acrylate, methacrylate or itaconate monomer, in particular acrylates/steareth-20 meth-acrylate copolymer, such as the acrylates/steareth-20 methacrylate copolymer (Aculyn 22), are used.

ii) The copolymers based on 2-acrylamido-2-methylpropanesulphonic acid can be selected from the copolymers of 2-acrylamido-2-methylpropanesulphonic acid and ethylenically unsaturated hydrophilic monomers selected for example from (meth)acrylic acids, β-substituted alkyl derivatives thereof or esters thereof obtained with monoalcohols or mono- or polyalkylene glycols, alkyl(meth)acrylamides, vinylpyrrolidone, vinylformamide, maleic anhydride, itaconic acid, maleic acid or mixtures of these compounds.

These polymers according to the invention can be crosslinked or non-crosslinked.

When the polymers are crosslinked, the crosslinking agents can be selected from the olefinically polyunsaturated compounds commonly used for the cross-linking of polymers obtained by radical polymerization.

As crosslinking agents, divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylene-bisacrylamide, methylene-bismethacrylamide, triallylamine, triallyl cyanurate, diallyl maleate, tetraallylethylenediamine, tetraallyloxyethane, tri-methylolpropane diallyl ether, allyl (meth)acrylate, allyl ethers of alcohols of the sugars series, or other allyl or vinyl ethers of polyfunctional alcohols, as well as allyl esters of derivatives of phosphoric and/or vinylphosphonic acids, or mixtures of these compounds, can for example be cited.

According to a preferred implementation mode of the invention, the crosslinking agent is selected from methylene-bisacrylamide, allyl methacrylate or tri-methylolpropane triacrylate (TMPTA). The crosslinking level in general ranges from 0.01 to 10% in moles and more particularly from 0.2 to 2% in moles relative to the polymer.

As the copolymer capable of being obtained from 2-acrylamido-2-methylpropanesulphonic acid and ethylenically unsaturated hydrophilic monomers, use can in particular be made of copolymers of 2-acrylamido-2-methylpropanesulphonic acid and alkyl (meth)acrylamides such as for example:

(1) anionic crosslinked copolymers of acrylamide or methylacrylamide and 2-acrylamido-2-methylpropane-sulphonic acid, in particular those taking the form of a W/O emulsion, such as those marketed under the name SEPIGEL 305 by Seppic (CTFA name: Polyacrylamide/C13-14 Isoparaffin/Laureth-7), and under the name SIMULGEL 600 by Seppic (CTFA name: Acrylamide/Sodium acryloyl-dimethyltaurate/Isohexadecane/Polysorbate 80 copolymer),
(2) copolymers of methacrylic acid or (meth)acrylate and 2-acrylamido-2-methyl-propanesulphonic acid, in particular those taking the form of a W/O emulsion, such as those marketed under the name SIMULGEL NS by Seppic (copolymer of sodium acrylamido-2-methyl-propanesulphonate/hydroxyethyl acrylate in 40% inverse emulsion in Polysorbate 60 and squalane) (CTFA name: hydroxyethyl acrylate/sodium acryloyldimethyl-taurate/squalane/polysorbate 60 copolymer) or those marketed under the name SIMULGEL EG by Seppic (copolymer of acrylic/acrylamido-2-methylpropane-sulphonic acid in the sodium salt form in 45% inverse emulsion in isohexadecane/water) (CTFA name: Sodium Acrylate/Sodium acryloydimethyltaurate copolymer/Isohexadecane/Polysorbate 80).

Preferably the composition A of the kit according to the invention contains at least one gelling agent selected from copolymers of 2-acrylamido-2-methyl-propanesulphonic acid, in particular copolymers of 2-acrylamido-2-methylpropanesulphonic acid and alkyl-(meth)acrylamide as described above.

Preferably the composition A of the kit according to the invention contains at least one gelling agent selected from copolymers of 2-acrylamido-2-methyl-propanesulphonic acid, in particular copolymers of 2-acrylamido-2-methylpropanesulphonic acid and alkyl-(meth)acrylamide taking the form of an inverse water-in-oil emulsion, the oil being in particular an alkane containing from 7 to 16 carbon atoms, for example a branched alkane such as isododecane or isohexadecane, the emulsion preferably containing a surfactant such as polyethoxylated sorbitan monooleate (20 EO).

### Surfactants

The composition A according to the invention can contain at least one surfactant (or emulsifier). The surfactants used are preferably surfactants capable of forming emulsions without application of heat and by simple manual stirring.

The surfactants can be present at a content ranging from 1% to 25% by weight, preferably from 3% to 20% by weight and still better from 5% to 15% by weight relative to the total weight of composition A.

The surfactants can be selected from the amphoteric, anionic, cationic or nonionic surfactants, used alone or mixed. The emulsifiers are selected appropriately depending on the continuous phase of the emulsion to be obtained (W/O or O/W).

As surfactants usable for the preparation of W/O emulsions, the alkyl esters or ethers of sorbitan, glycerol or sugars can for example be cited.

To this can also be added one or more co-emulsifiers, which can advantageously be selected from the group comprising esters of a branched chain fatty acid and a polyol, and in particular esters of a branched chain fatty acid and glycerol and/or sorbitan and for example polyglyceryl isostearate, such as the product marketed under the name Isolan GI 34 by Goldschmidt, sorbitan isostearate, such as the product marketed under the name Arlacel 987 by ICI, sorbitan and glycerol isostearate, such as the product marketed under the name Arlacel 986 by ICI, and mixtures thereof.

For the O/W emulsions, as emulsifiers, nonionic surfactants, and in particular esters of polyols and a saturated or unsaturated chain fatty acid containing for example from 8 to 24 carbon atoms and better from 12 to 22 carbon atoms, and alkoxylated derivatives thereof, in other words containing ethoxylated and/or propoxylated units, such as the glyceryl esters of C₈-C₂₄ fatty acids and alkoxylated derivatives thereof, polyethylene glycol esters of C₈-C₂₄ fatty acids, and alkoxylated derivatives thereof, sorbitol esters of C₈-C₂₄ fatty acids, and alkoxylated derivatives thereof, sugar (sucrose, glucose, alkylglucose) esters of C₈-C₂₄ fatty acids, and alkoxylated derivatives thereof, ethers of fatty alcohols, ethers of sugar and C₈-C₂₄ fatty alcohols, and mixtures thereof can for example be cited.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

As polyethylene glycol esters of fatty acids, poly-ethylene glycol stearate (polyethylene glycol mono-, di- and/or tristearate), and more specifically poly-ethylene glycol 50 EO monostearate (CTFA name: PEG-50 stearate), polyethylene glycol 100 EO monostearate (CTFA name: PEG-100 stearate) and mixtures thereof can in particular be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Ubiqema.

As esters of fatty acids and glucose or alkylglucose, in particular glucose palmitate, alkylglucose sesquistearates such as methylglucose sesquistearate, alkylglucose palmitates such as methylglucose or ethylglucose palmitate, methylglucoside fatty esters and more specifically the diester of methylglucoside and oleic acid (CTFA name: Methyl glucose dioleate), the mixed ester of methylglucoside and the mixture oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As ethers of fatty alcohols, ethers of polyethylene glycol and a fatty alcohol containing from 8 to 30 carbon atoms, and in particular from 10 to 22 carbon atoms, such as the ethers of polyethylene glycol and cetyl, stearyl and cetearyl alcohols (mixture of cetyl and stearyl alcohols) can for example be cited. Ethers containing from 1 to 200 and preferably from 2 to 100 ethoxylate groups, such as those of CTFA name Ceteareth-20, Ceteareth-30, and mixtures thereof can for example be cited.

As sugar ethers, alkylpolyglucosides, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

Co-emulsifiers such as for example fatty alcohols having from 8 to 26 carbon atoms, such as cetyl alcohol, stearyl alcohol and the mixture thereof (cetearyl alcohol), octyl dodecanol, 2-butyloctanol, 2-hexyldecanol, 2-undecylpentadecanol or oleyl alcohol, or fatty acids, can be added to these emulsifiers.

According to one implementation mode, the composition A contains at least one surfactant selected from esters of sucrose, esters of fatty acids and alkoxylated sorbitan containing for example from 20 to 100 EO, esters of fatty acids and glucose or alkylglucose, such as alkylglucose sesquistearates, ethoxylated ethers of fatty acids and glucose or alkylglucoses such as ethoxylated ethers of fatty acids and methylglucose, mixtures of glycerides of alkoxylated plant oils such as mixtures of ethoxylated (200 EO) palm and copra (7 EO) glycerides, and mixtures thereof.

### Lipophilic Structuring Agent

According to one implementation mode, the composition A advantageously contains at least one lipophilic structuring agent (compound distinct from the gelling agent).

The lipophilic structuring agent is selected from the polymeric structuring agents.

In the sense of the invention, polymer is understood to mean a compound having at least 2 repeating moieties, preferably at least 3 repeating moieties and better still 10 repeating moieties.

The quantity (as active ingredient) of lipophilic structuring agent can range from 0.5% to 20% by weight, preferably from 1% to 15% by weight and still better from 2% to 10% by weight relative to the total weight of composition A.

The polymeric structuring agents can in particular be selected from a) polyamide polymers, b) olefin copolymers, c) semi-crystalline polymers, d) dextrin esters of fatty acids, e) pyrogenic silicas and f) organophilic clays and mixtures thereof.

### a) Polyamides

The particular polyamides used in the composition according to the present invention are preferably those described in the document U.S. Pat. No. 5,783,657 from UNION CAMP. The part of U.S. Pat. No. 5,783,657 devoted to these polymers is incorporated by reference.

Each of these polyamides in particular satisfies the following formula:

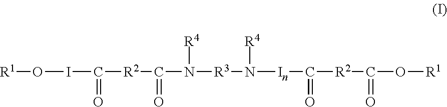

in which n designates a whole number of amide moieties such that the number of ester groups represents from 10% to 50% of the total number of ester and amide groups, at each occurrence, R¹ is independently an alkyl or alkenyl group having at least 4 carbon atoms and in particular from 4 to 24 carbon atoms, at each occurrence R² independently represents a C₄ to C₅₅ hydrocarbon group on condition that 50% at least of the R² groups represent a C₃₀ to C₅₅ hydrocarbon group, at each occurrence R³ independently represents an organic group having at least 2 carbon atoms, hydrogen atoms and optionally one or more oxygen or nitrogen atoms, and at each occurrence R⁴ independently represents a hydrogen atom, C₁ to C₁₀ alkyl group or a direct bond to R³ or to another R⁴ such that the nitrogen atom to which both R³ and R⁴ are bound forms part of a heterocyclic structure defined by R⁴—N—R³, with at least 50% of the R⁴ representing a hydrogen atom. In particular, the ester groups of this polyamide represent from 15 to 40% of the total number of ester and amide groups and at best from 20 to 35%. Further, n advantageously represents a whole number ranging from 1 to 10, and better from 1 to 5, inclusive of termini.

Preferably, R¹ is a C₁₂ to C₂₂ and preferably C₁₆ to C₂₂ alkyl group. Advantageously, R² can be a C₁₀ to C₄₂ hydrocarbon (alkylene) group. Preferably, 50% at least and better 75% at least of the R² are groups having from 30 to 42 carbon atoms. The other R² are hydrogenated C₄ to C₁₉ and preferably C₄ to C₁₂ groups. Preferably, R³ represents a C₂ to C₃₆ hydrocarbon group or a polyalkoxylated group and R⁴ represents a hydrogen atom. Preferably, R³ represents a C₂ to C₁₂ hydrocarbon group. The hydrocarbon groups can be linear, cyclic or branched, saturated or unsaturated groups. Moreover, the alkyl and alkylene groups can be linear or branched, saturated or unsaturated groups.

In general, these polyamides take the form of mixtures, and these mixtures can further contain a synthetic product corresponding to a polyamide as defined above with n equal to 0, in other words a diester.

As structuring polyamides usable in the invention, polyamide resins resulting from the condensation of an aliphatic dicarboxylic acid and a diamine (including compounds having more than two carbonyl groups and two amine groups), the carbonyl and amine groups of adjacent unit moieties being condensed by an amide bond, can also be cited. These polyamide resins are in particular those marketed under the trade name Versamid® by General Mills, Inc. and Henkel Corp., under the trade name Onamid® in particular Onamid S or C. These resins have a weight average molecular weight ranging from 6000 and 9000. For more information on these polyamides, reference can be made to the documents U.S. Pat. No. 3,645,705 and U.S. Pat. No. 3,148,125. More specifically, Versamid® 30 or 744 are used.

The polyamides sold or produced by Arizona under the references Uni-Rez (2658, 2931, 2970, 2621, 2613, 2624, 2665, 1554, 2623 and 2662) and the product sold under the reference Macromelt 6212 by Henkel can also be used. For more information on these polyamides, reference can be made to the document USA-5500209.

As examples of structuring polyamides usable in the composition according to the invention, the commercial products sold or produced by Arizona Chemical under the names Uniclear 80 and Uniclear 100 VG can also be cited. They are sold respectively in the form of an 80% (active ingredient) gel and 100% (active ingredient) in a mineral oil. They have a softening point of 88 to 105° C. These commercial products are a mixture of a copolymer of a C36 diacid condensed with ethylene diamine, of average molecular weight about 6000. The terminal ester groups result from the esterification of the residual acid termini by cetyl or stearyl alcohol or mixtures thereof (also called cetylstearyl alcohol).

b) Olefin copolymer

In the sense of the present application, olefin copolymer is understood to mean any copolymer formed by polymerization of at least one olefin and another additional monomer different from the said olefin.

The olefin can in particular be an ethylenically unsaturated monomer.

As examples of olefins, ethylenic hydrocarbon monomers, having in particular one or two ethylenic unsaturations, and having from 2 to 5 carbon atoms such as ethylene, propylene, butadiene or isoprene, can be cited.

The olefin copolymer is preferably selected from the amorphous olefin copolymers.

Amorphous polymer is understood to mean a polymer which does not have a crystalline form. This polymer can also be filmogenic, in other words it is capable of forming a film when it is applied onto the skin.

The olefin copolymer can in particular be a diblock, triblock, multiblock, radial or star copolymer, or mixtures thereof.

Such compounds are for example described in the application US-A-2002/005562 and in the U.S. Pat. No. 5,221,534

An amorphous block copolymer of styrene and an olefin is advantageously selected.

The block copolymer is preferably hydrogenated to reduce the residual ethylenic unsaturations after the polymerization of the monomers.

In particular, the hydrocarbon block copolymer is a copolymer, possibly hydrogenated, with styrene and C₃-C₄ ethylene/alkylene blocks.

As diblock copolymers, preferably hydrogenated, styrene-ethylene/propylene copolymers, styrene-ethylene/butadiene copolymers, and styrene-ethylene/butylene copolymers can be cited. Diblock copolymers are in particular sold under the name Kraton® G1701E by Kraton Polymers.

As triblock copolymers, preferably hydrogenated, styrene-ethylene/propylene-styrene copolymers, styrene-ethylene/butadiene-styrene copolymers, styrene-isoprene-styrene copolymers, and styrene-butadiene-styrene copolymers can be cited.

Triblock polymers are in particular sold under the names Kraton® G1650E, Kraton® G1652, Kraton® D1101, Kraton® D1102 and Kraton® D1160 by Kraton Polymers. A styrene-ethylene/butylene-styrene triblock copolymer can in particular be used.

According to one implementation mode of the invention, a mixture of a styrene-butylene/ethylene-styrene triblock copolymer and a styrene-ethylene/butylene diblock copolymer, in particular sold under the name Kraton® G1657M by Kraton Polymers, can in particular be used.

A mixture of hydrogenated styrene-butylene/ethylene-styrene triblock copolymer and hydrogenated ethylene-propylene-styrene star copolymer, such a mixture being in particular in isododecane can also be used. Such mixtures are for example sold by PENRECO under the trade names VERSAGEL® M5960 and VERSAGEL® M5670.

c) Semi-crystalline polymers

The semi-crystalline polymers are preferably derivatives of acrylic or methacrylic acid. In the sense of the invention, “semi-crystalline polymer” is understood to mean polymers containing a crystallisable part, hanging chain or sequence in the skeleton, and an amorphous part in the skeleton, and exhibiting a first order reversible phase change, in particular melting (solid-liquid transition), temperature. When the crystallisable part is a sequence of the polymeric skeleton, this crystallisable sequence is different in chemical nature from that of the amorphous sequences; in this case the semi-crystalline polymer is a block copolymer for example of the diblock, triblock or multiblock type.

Advantageously, the semi-crystalline polymer or polymers of the composition of the invention have a number average molecular weight Mn greater than or equal to 2000, ranging for example from 2,000 to 800,000, preferably from 3,000 to 500,000, for example from 4,000 to 150,000, and better from 4,000 to 99,000.

In the composition according to the invention, the semi-crystalline polymers are advantageously at least 1% by weight soluble in the oily phase, at a temperature greater than their melting point. Outside the crystallisable chains or sequences, the sequences of the polymers are amorphous. In the sense of the invention, “crystallisable chain or sequence” is understood to mean a chain or sequence which, if it were alone, would pass from the amorphous state to the crystalline state, reversibly, depending on whether it is above or below the melting point. A chain in the sense of the invention is a group of atoms, hanging or lateral relative to the skeleton of the polymer. A sequence is a group of atoms belonging to the skeleton, a group constituting one of the repetitive moieties of the polymer.

Preferably, the polymeric skeleton of the semi-crystalline polymers is soluble in the oily phase. Preferably, the semi-crystalline polymers used in the composition of the invention exhibit a melting temperature (or melting point), M.Pt., lower than 70° C. (25° C.≦M.Pt.<70° C.), that temperature being at least equal to the temperature of the keratinous material intended to receive the composition according to the invention, in particular the skin. The melting point can in particular be measured by any known method and in particular with a differential scanning calorimeter (D.S.C).

Preferably, the crystallisable sequences or chains of the semi-crystalline polymers represent at least 30% of the total weight of each polymer and better at least 40%. The semi-crystalline polymers with crystallisable sequences used according to the invention are block or multiblock copolymers. They can be obtained by polymerization of monomers with reactive (or ethylenic) double bonds or by polycondensation. When the polymers of the invention are polymers with crystallisable side chains, they are advantageously in random or statistical form.

The semi-crystalline polymers of the invention are of synthetic origin. Further, they do not contain a poly-saccharide skeleton.

The semi-crystalline polymers usable in the invention are preferably selected from polymers (homopolymers or copolymers) bearing at least one crystallisable side chain, and polymers (homopolymers or copolymers) bearing in the skeleton at least one crystallisable sequence, such as those described in the document U.S. Pat. No. 5,156,911. The crystallisable side chain(s) or sequence(s) are hydrophobic.

According to a preferred implementation mode of the invention, the semi-crystalline polymers are selected in particular from homopolymers and copolymers resulting from the polymerization of at least one monomer with crystallisable chain(s), the latter being selected from alkyl chains containing at least 11 carbon atoms and at most 40 carbon atoms and better at most 24 carbon atoms. These are in particular alkyl chains containing at least 12 carbon atoms, and preferably these are alkyl chains containing from 14 to 24 carbon atoms (C₁₄-C₂₄). They can be hydrocarbon alkyl chains (carbon atoms and hydrogen) or fluorinated or perfluorinated alkyl chains (carbon atoms, fluorine atoms and possibly hydrogen atoms). When they are fluorinated or perfluorinated alkyl chains, they contain at least 11 carbon atoms of which at least 6 carbon atoms are fluorinated.

In the sense of the invention, “alkyl” is understood to mean a saturated group (containing no unsaturation).

According to a particular implementation mode of the invention, the semi-crystalline polymer is selected from homopolymers obtained by polymerization of at least one monomer with a crystallisable chain, selected from C₁₄-C₂₄ alkyl (meth)acrylates, perfluoroalkyl (meth)acrylates, C₁₄ to C₂₄ N alkyl (meth)acrylamides with or without a fluorine atom, vinyl esters with C₁₄ to C₂₄ alkyl or perfluoroalkyl chains, vinyl ethers with C₁₄ to C₂₄ alkyl or perfluoroalkyl chains, C₁₄ to C₂₄ alpha-olefins, para-alkyl styrenes with a C₁₄ to C₂₄ alkyl group, and from copolymers of these monomers, obtained by copolymerization of these monomers with a hydrophilic monomer, preferably different from methacrylic acid, such as for example N-vinyl-pyrrolidone, hydroxyethyl acrylate, hydroxyethyl methacrylate or acrylic acid. Such copolymers can for example be copolymers of C₁₄-C₂₄ alkyl acrylate, C₁₄-C₂₄ alkyl methacrylate, C₁₄-C₂₄ alkyl acrylamide or C₁₄-C₂₄ alkyl methacrylamide with N-vinylpyrrolidone, hydroxy-ethyl acrylate, hydroxyethyl methacrylate, acrylic acid, or mixtures thereof.

Preferably, the semi-crystalline polymer is selected from homopolymers obtained by polymerization of a monomer selected from C₁₄-C₂₄ alkyl acrylates and C₁₄-C₂₄ alkyl methacrylates and from copolymers obtained by copolymerization of a monomer selected from C₁₄-C₂₄ alkyl acrylates and C₁₄-C₂₄ alkyl methacrylates, with a hydrophilic monomer such as acrylic acid.

The semi-crystalline polymers of the composition of the invention can be non-crosslinked or crosslinked in part, provided that the degree of crosslinking does not hinder their dissolution or dispersion in the oily phase by heating above their melting point. This can then be a chemical crosslinking, by reaction with a multifunctional monomer during the polymerization. It can also be a physical crosslinking which can then be due either to the establishment of hydrogen type or dipolar bonds between groups borne by the polymer, such as for example dipolar interactions between carboxylate ionomers, these interactions being in small amount and borne by the skeleton of the polymer, or to a phase separation between the crystallisable sequences and the amorphous sequences borne by the polymer.

Preferably, the semi-crystalline polymers of the composition according to the invention are non-crosslinked.

According to a particular implementation mode of the invention, the semi-crystalline polymer is a homopolymer resulting from the polymerization of a monomer with a crystallisable chain selected from C₁₄-C₂₄ alkyl acrylates and C₁₄-C₂₄ alkyl methacrylates. Those marketed under the names Intelimer® by Landec, described in the brochure “Intelimer® polymers”, Landec IP22 can in particular be cited. These polymers are in solid form at ambient temperature. They bear crystallisable side chains and correspond to homopolymers of saturated C₁₄-C₂₄ alkyl acrylates or methacrylates. More particularly, the homopolymer of stearyl acrylate (Intelimer IPA-13.1) (INCI name: Poly C10-30 alkyl acrylate), and the homopolymer of behenyl acrylate (Intelimer IPA-13.6) (INCI name: Poly C10-30 alkyl acrylate) can be cited.

According to another particular implementation mode of the invention, the semi-crystalline polymer is a copolymer of C₁₄-C₂₄ alkyl acrylates or C₁₄-C₂₄ alkyl methacrylates with acrylic acid. As copolymers of this type, the copolymers obtained by copolymerization of behenyl acrylate and acrylic acid, and the copolymers obtained by copolymerization of stearyl acrylate and acrylic acid can be cited.

According to a preferred implementation mode of the invention, the semi-crystalline polymer is a homopolymer, and it is selected from the homopolymer of stearyl acrylate (Intelimer IPA-13.1) (INCI name: Poly C10-30 alkyl acrylate), the homopolymer of behenyl acrylate (Intelimer IPA-13.6) (INCI name: Poly C10-30 alkyl acrylate), and mixtures thereof.

d) Ester of dextrin and fatty acid(s)

The composition according to the invention contains at least one ester of dextrin and fatty acid(s).

More particularly, this is a mono- or polyester of dextrin and at least one fatty acid and in particular corresponding to the formula (C):

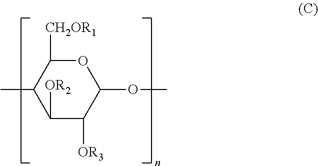

wherein:
n is a whole number ranging from 3 to 200, in particular ranging from 20 to 150, and in particular ranging from 25 to 50,
the radicals R₁, R₂ and R₃, identical or different, are selected from hydrogen or an acyl group (R—CO—) wherein the radical R is a linear or branched, saturated or unsaturated hydrocarbon group, having from 5 to 29, in particular from 7 to 21, especially from 11 to 19, more particularly from 13 to 17, or even 15, carbon atoms, provided that at least one of the said radicals R₁, R₂ or R₃ is different from hydrogen.

In particular, R₁, R₂ and R₃ can represent hydrogen or an acyl group (R—CO—) wherein R is a hydrocarbon radical as previously defined, provided that at least two of the said radicals R₁, R₂ or R₃ are identical and different from hydrogen.

The set of radicals R₁, R₂ and R₃ can feature an identical or different, and in particular identical, acyl group (R—CO).

In particular, n advantageously varies from 25 to 50, and in particular equals 38 in the general formula (C) of the ester according to the invention.

In particular when the radicals R₁, R₂ and/or R₃, identical or different, feature an acyl group (R—CO), these can be selected from the caprylic, capric, lauric, myristic, palmitic, stearic, arachic, behenic, isobutyric, isovaleric, 2-ethylbutyric, ethylmethyl-acetic, isoheptanoic, 2-ethylhexanoic, isononanoic, isodecanoic, isotridecanoic, isomyristic, isopalmitic, isostearic, isoaracic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic, stearolic, eicosanyl and docosanoyl radicals, and mixtures thereof.

Preferably, as the ester of dextrin and fatty acid(s) at least one dextrin palmitate is used. This can be used alone or mixed with other esters.

Advantageously, the ester of dextrin and fatty acid has a degree of substitution less than or equal to 2.5 on the basis of one glucose unit, in particular varying from 1.5 to 2.5, preferably from 2 to 2.5. The weight average molecular weight of the dextrin ester can in particular be from 10,000 to 150,000, in particular from 12,000 to 100,000 and even from 15,000 to 80,000 Dextrin esters, in particular dextrin palmitates, are commercially available under the name RHEOPEARL TL or RHEOPEARL KL from Chiba Flour.

### e) Pyrogenic Silicas

Pyrogenic silicas are obtained by continuous flame pyrolysis of silicon tetrachloride (SiCl₄) at 1000° C. in the presence of hydrogen and oxygen. They can be hydrophilic or hydrophobic.

In the present application, “hydrophilic silica” is understood to mean both pure hydrophilic silicas and particles coated with hydrophilic silica.

In the present application, “hydrophobic silica” is understood to mean both pure hydrophobic silicas and particles coated with hydrophobic silica.

Among the pyrogenic silicas of hydrophilic character usable according to the present invention, those sold by DEGUSSA HÜLS under the trade names AEROSIL 90, 130, 150, 200, 300 and 380 can in particular be cited.

Among the pyrogenic silicas of hydrophobic character usable according to the present invention, those sold by DEGUSSA HÜLS under the trade names AEROSIL® R202, R805, R812, R972 and R974 can in particular be cited.

### f) Organophilic Clays

As organophilic clays, modified clays such as hectorites modified with a C₁₀ to C₂₂ ammonium chloride, such as hectorite modified with distearyl dimethyl ammonium chloride such as, for example, that marketed under the name Bentone 38V® by ELEMENTIS can be cited.

The structuring agent is preferably selected from the esters of dextrin and fatty acid(s), in particular dextrin palmitate.

### Polyols

The composition A advantageously contains a polyol which can be selected from glycerol, sorbitol, propylene glycol, pentylene glycol, butylene glycol, propanediol, products of addition of ethylene oxide and C3-C4 alkylene oxide to a polyol such as glycerol such as for example polyoxybutylene polyoxyethylene polyoxy-propylene glycerol (INCI name PEG/PPG/polybutylene glycol-8/5/3 glycerine) such as the compound marketed under the name WILBRIDE S-753 by NOF, the 2-ethylhexyl ester of glycerol (INCI name ethyl hexyl glycerine), caprylyl glycol and mixtures thereof.

The polyols can represent from 5% to 40% by weight, better from 10% to 30% by weight and still better from 12% to 25% by weight of the composition A.

### B. Aqueous Composition

In the present application, “aqueous composition” is understood to mean a composition containing at least some water.

The aqueous composition, apart from water, can contain an organic solvent soluble in water, selected for example from the lower mono-alcohols containing from 1 to 8 carbon atoms and in particular 1 to 6 carbon atoms, such as ethanol, isopropanol, propanol and butanol, polyethylene glycols having from 6 to 80 ethylene oxide groups, polyols such as propylene glycol, isoprene glycol, butylene glycol, glycerine and sorbitol, acetone, and mixtures thereof.

Preferably, composition B is not an emulsion. Preferably, composition B is an aqueous solution. According to an embodiment, composition B contains only water and possibly at least one water-soluble organic solvent, which means that water and organic solvent soluble in water, if present, represent 100% of composition B. More specifically, composition B contains only water and a water-soluble organic solvent.

Preferably again, composition B is free from fatty substances, in particular oils.

The water used in the composition of the invention can be pure demineralized water but also mineral water and/or thermal water and/or sea water, in other words the water of the composition can be partially or wholly constituted of a water selected from mineral waters, thermal waters, sea waters and mixtures thereof. Mineral or thermal waters will refer not only to natural mineral waters or thermal waters, but also to natural or thermal waters enriched in mineral constituents and/or supplementary oligoelements, as well as inorganic aqueous solutions and/or those containing oligoelements prepared from purified (demineralized or distilled) water.

A thermal or natural mineral water used according to the invention can, for example, be selected from Vittel water, Vichy Basin waters, Uriage water, Roche Posay water, Bourboule water, Enghien-les-Bains water, Saint Gervais-les-Bains water, Neris-les-Bains water, Allevar-les-Bains water, Digne water, Maizières water, Neyrac-les-Bains water, Lons-le-Saunier water, les Eaux Bonnes, Rochefort water, Saint Christau water, Fumades water and Tercis-les-bains water, and Avene water.

Preferably, composition B contains at least 70%, preferably at least 75% and better at least 80% water.

### C/ Additives

Composition A and/or composition B, preferably composition A, can contain at least one additive which can in particular be selected from cosmetic active ingredients such as hydrating active ingredients, anti-seborrhoeic agents, anti-ageing active ingredients, anti-wrinkle active ingredients, antimicrobial active ingredients, anti-inflammatory or soothing active ingredients, lipolytic or slimming active ingredients, fillers, sunscreens, agents for dyeing the skin or hair, concealer active ingredients, anti-perspirant active ingredients, deodorant ingredients, hair treatment active ingredients, depilatory agents, pigments, colorants, perfumes, electrolytes, pH adjusters, preservatives, and mixtures thereof.

I) Hydrating active ingredients, such as for example sodium lactate, polyols, and in particular glycerine, sorbitol or polyethylene glycols, mannitol, amino acids, hyaluronic acid and derivatives thereof, lanolin, urea and mixtures containing urea, such as NMF (“Natural Moisturizing Factor”), vaseline, N-lauroyl pyrrolidone carboxylic acid and salts thereof, essential fatty acids, essential oils, and mixtures thereof.
II) Anti-seborrheic agents, selected for example from:
sulphur and sulphur derivatives;
zinc salts such as zinc lactate, gluconate, pidolate, carboxylate, salicylate and/or cysteate;
selenium chloride;
vitamin B6 or pyridoxine;
the mixture of capryloyl glycine, sarcosine and extract of Cinnamomum zeylanicum marketed in particular by SEPPIC under the trade name Sepicontrol A5®;
an extract of Laminaria saccharina marketed in particular by SECMA under the trade name Phlorogine®;
an extract of Spiraea ulmaria marketed in particular by SILAB under the trade name Sebonormine®;
extracts of plants of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all marketed for example by MARUZEN;
an extract of Serenoa repens marketed in particular by EUROMED;
extracts of plants of the Silybum genus;
plant extracts containing sapogenins and in particular extracts of Dioscoreaceae rich in diosgenin or hecogenin;
extracts of Eugenia caryophyllata containing eugenol and eugenyl glucoside;
and mixtures thereof.
   III) The anti-ageing or anti-wrinkle active ingredients can be selected from all active ingredients capable of treating or preventing any sign of ageing of the skin. They can for example be selected from anti-free radical agents, keratolytic agents, vitamins, anti-elastase and anti-collagenase agents, protides, fatty acid derivatives, steroids, oligo-elements, whiteners, extracts of algae and plankton, enzymes and coenzymes, flavonoids, ceramides, toning agents, myorelaxant agents, sugars, and mixtures thereof.
   1) As anti-free radical and anti-oxidant agents, derivatives of phosphonic acid such as ethylene diamine tetra(methylene phosphonic) acid, hexamethylene diamine tetra(methylene phosphonic) acid, diethylene triamine penta(methylenephosphonic) acid, and salts thereof and in particular sodium salts thereof, ethylene diamine tetraacetic acid and salts thereof such as the sodium salt, guanosine, superoxidismutase, tocopherol (vitamin E) and derivatives thereof (acetate), ethoxyquin, lactoferrin, lactoperoxidase, and nitroxide derivatives, superoxide dismutases, glutathione peroxidase, plant extracts with anti-radical activity such as the aqueous wheat germ extract marketed by Silab under the reference Detoxiline, plant extracts rich in polyphenols such as green tea and grape, and mixtures thereof can in particular be cited.
   2) As keratolytic agents, α-hydroxy-acids in particular acids derived from fruit, such as glycolic, lactic, malic, citric, tartaric and mandelic acids, derivatives thereof and mixtures thereof, β-hydroxy-acids such as salicylic acid and derivatives thereof such as n-octanoyl-5-salicylic acid or n-dodecanoyl-5-salicylic acid, α-keto-acids such as ascorbic acid or vitamin C and derivatives thereof such as salts thereof such as sodium ascorbate, magnesium or sodium ascorbylphosphate, esters thereof such as ascorbyl acetate, ascorbyl palmitate and ascorbyl propionate, or sugar derivatives thereof such as glycosylated ascorbic acid, and mixtures thereof, β-keto-acids, retinoids such as retinol (vitamin A) and esters thereof, retinal, retinoic acid and derivatives thereof, as well as the retinoids described in the documents FR-A-2,570,377, EP-A-199636, EP-A-325540, EP-A-402072, adapalene, carotenoids, and mixtures thereof can for example be cited.
   3) As vitamins, apart from the aforesaid vitamins A, E and C, vitamin B3 (or vitamin PP or niacinamide) and derivatives thereof (tocopherol nicotinate, esters of nicotinyl alcohol and carboxylic acids, 2-chloro-nicotinamide, 6-methylnicotinamide, 6-aminonicotin-amide, N-methylnicotinamide, N,N-dimethylnicotinamide, N-(hydroxymethyl)nicotinamide, quinolinic acid imide, nicotinanilide, N-benzylnicotinamide, N-ethylnicotin-amide, nifenazone, nicotinaldehyde, isonicotinic acid, methylisonicotinic acid, thionicotinamide, nialamide, 2-mercaptonicotinic acid, nicomol and niaprazine), vitamin B5 (or panthenol or panthenyl alcohol or 2,4-dihydroxy-N-(3-hydroxypropyl)-3,3-dimethyl-butanamide), in its various forms: D-panthenol, DL-panthenol), and derivatives and analogues thereof, such as calcium pantothenate, panthetine, pantotheine, ethyl panthenyl ether, pangamic acid, pyridoxine, pantoyl lactose, and natural compounds containing these such as royal jelly, vitamin D and analogues thereof such as those described in the document WO-A-00/26167; vitamin F or analogues thereof such as mixtures of unsaturated acids having at least one double bond and in particular mixtures of linoleic acid, linolenic acid and arachidonic acid, or compounds containing these and in particular oils of plant origin containing these, such as for example jojoba oil, and mixtures thereof can in particular be cited.
   4) As anti-elastase agents, peptide derivatives, and in particular the peptides from the grains of leguminous plants such as those marketed by Laboratoires Sériobiologiques de Nancy under the reference Parelastyl; the derivatives of N-acylamino-amides described in the application FR-A-2,180,033, such as for example ethyl {2-[acetyl-(3-trifluoromethylphenyl)-amino]-3-methyl-butyrylamino}acetate and {2-[acetyl-(3-trifluoro-methylphenyl)-amino]-3-methyl-butyrylamino}-acetic acid and mixtures thereof can in particular be cited.
   5) As anti-collagenase agents, metalloprotease inhibitors, such as ethylenediamine-tetraacetic acid (EDTA), cysteine, and mixtures thereof can be cited.
   6) As protides (or peptides), the proteins of wheat, rice, malt or soya, and hydrolysates thereof, such as those marketed by Silab under the reference Tensine, and mixtures thereof can for example be cited.
   7) As fatty acid derivatives, polyunsaturated phospholipids including the phospholipids of essential fatty acids of soya, and mixtures thereof can in particular be cited.
   8) As steroids, DHEA or dehydroepi-androsterone, biological precursors thereof, metabolites thereof, and mixtures thereof can for example be cited. “Biological precursors” of DHEA is understood to mean in particular Δ5-pregnenolone, 17α-hydroxy pregnenolone and 17α-hydroxy pregnenolone sulphate. Derivatives of DHEA, is understood to mean both metabolic derivatives thereof and chemical derivatives thereof. As metabolic derivatives, Δ5-androstene-3,17-diol and in particular 5-androstene 3β, 17β-diol, Δ4-androstene-3,17-dione, 7 hydroxy DHEA (7α-hydroxy DHEA or 7β-hydroxy-DHEA) and 7-keto-DHEA which is itself a metabolite of 7β-hydroxy DHEA and benzoyl DHEA can in particular be cited.
   9) As oligoelements, copper, zinc, selenium, iron, magnesium, manganese and mixtures thereof can for example be cited.
   10) As whiteners or depigmenting agents, kojic acid and derivatives thereof, hydroquinone and derivatives thereof such as arbutine and esters thereof, vitamin C and derivatives thereof such as magnesium ascorbyl phosphate, salts such as calcium D pantetheine sulphonate, ellagic acid and derivatives thereof, rucinol, linoleic acid and derivatives thereof, extracts of plants and in particular of liquorice, mulberry or scutellaria, glutathione and precursors thereof, cysteine and precursors thereof, the compounds derived from aminophenol described in the document WO-A-99/10318, such as in particular N-ethyl-oxycarbonyl-4-aminophenol, N-ethyloxycarbonyl-O-ethyloxycarbonyl-4-aminophenol, N-cholesteryloxycarbonyl-4-aminophenol, N-ethylaminocarbonyl-4-aminophenol, phenyl-ethyl-resorcinol (Symwhite) and mixtures of these compounds can for example be cited.
   11) As extracts of algae, extracts of red or brown algae, and for example the extract of brown algae of the Laminaria family, such as extracts from the species Laminaria digitata, and more particularly that sold by CODIF under the name Phycosaccharides, which is a concentrated solution of an oligosaccharide comprising the concatenation of two uronic acids, mannuronic acid and guluronic acid, can be cited.
   12) As plankton extracts, the plankton in aqueous dispersion (CTFA name: Vitreoscilla Ferment) marketed under the name MEXORYL SAH by Chimex can be cited.
   13) As enzymes, any enzyme of animal, microbiological (bacterial, fungal or viral) or synthetic (obtained by chemical or biotechnological synthesis) origin, in pure crystalline form or in a dilute form in an inert diluent can be used. Lipases, proteases, phospho-lipases, laccases, cellulases, peroxidases in particular lactoperoxidases, catalases, superoxide dismutases, or plant extracts containing the aforesaid enzymes, and mixtures thereof can for example be cited. They can for example be selected from that sold under the trade name “Subtilisine SP 554” by Novo Nordisk and that sold under the trade name “LYSOVEG LS” by Laboratoires Sérobiologiques de Nancy.
   14) As coenzymes, ubiquinone or coenzyme Q10 which belongs to the family of the benzoquinones with an alkenyl chain, coenzyme R which is biotin (or vitamin H), and mixtures thereof can in particular be used.
   15) As flavonoids, the isoflavonoids which constitute a subclass of the flavonoids, formed of a 3-phenyl chromane skeleton which can contain varied substituents and different oxidation levels can for example be cited. The term “isoflavonoid” brings together several classes of compounds among which the isoflavones, isoflavanones, rotenoids, pterocarpans, isoflavanes, isoflavane-3-enes, 3-arylcoumarins, 3-aryl-4-hydroxy-oumarins, coumestanes, coumaronochromones, α-methyl-deoxybenzoins, 2-arylbenzofurans, and mixtures thereof can be cited. The isoflavonoids can be of natural or synthetic origin. “Natural origin” is understood to mean the isoflavonoid in the pure state or in solution at various concentrations, obtained by various extraction processes from a constituent of natural origin, generally a plant. “Synthetic origin” is understood to mean the isoflavonoid in the pure state or in solution at different concentrations, obtained by chemical synthesis. As isoflavonoids of natural origin, daidzine, genistine, daidzeine, formononetine, cuneatine, genisteine, isoprunetine and prunetine, cajanine, orobol, pratenseine, santal, junipegenine A, glyciteine, afrormosine, retusine, tectorigenin, irisolidone and jamaicine, as well as analogues and metabolites thereof can be cited.
   16) As ceramides, any type of ceramide, of natural or synthetic origin, for example type II, type III, type IV, type V or type VI, and mixtures thereof can be used. As ceramides, N-oleoyldihydrosphingosine, N-stearoylphytosphingosine, N-α-hydroxybehenoyldihydro-sphingosine, N-α-hydroxypalmitoyldihydrosphingosine, N-linoleoyldihydrosphingosine, N-palmitoyldihydro-sphingosine, N-stearoyldihydrosphingosine, N-behenoyl-dihydrosphingosine, and mixtures thereof can for example be cited.
   17) As toning agents, the following can for example be cited:
synthetic polymers;
polymers of natural origin;
mixed silicates;
microparticles of wax;
colloidal particles of inorganic fillers.
The synthetic polymers usable as toning agents can be selected from:
   polyurethane polymers and copolymers;
   acrylic polymers and copolymers;
   polymers of sulphonated isophthalic acid;
   grafted silicone polymers;
   water-soluble or water-dispersible polymers containing water-soluble or water-dispersible units and LCST (Lower Critical Solution Temperature) units.
The polyurethane copolymers, acrylic copolymers and the other synthetic polymers capable of being used as toning agents can in particular be selected from poly-condensates, hybrid polymers and interpenetrated polymer networks (IPNs). “Inter-penetrated polymer networks” is understood to mean a mixture of two entangled polymers, obtained by simultaneous polymerization and/or crosslinking of two types of monomer, the mixture obtained having a unique glass transition temperature. Examples of IPNs suitable as toning polymers, and the process for preparation thereof, are for example described in the documents U.S. Pat. No. 6,139,322 and U.S. Pat. No. 6,465,001. Preferably, the IPN contains at least one polyacrylic polymer and, more preferably, it further contains at least one polyurethane or a copolymer of vinylidene fluoride and hexafluoropropylene. According to a preferred embodiment, the IPN comprises a polyurethane polymer and a polyacrylic polymer. Such IPNs are in particular those of the Hybridur range marketed by Air Products. An IPN particularly preferred as a toning polymer is in the form of an aqueous dispersion of particles having a weight average size lying between 90 and 110 nm and a number average size of about 80 nm. This IPN preferably has a glass transition temperature, Tg, which ranges from about −60° C. to +100° C. An IPN of this type is in particular marketed by Air Products under the trade name Hybridur X-01602. Another IPN suitable for use in the present invention is referred to as Hybridur X18693-21 or Hybridur 875 polymer dispersion.
Other IPNs suitable as toning polymers include the IPNs constituted of the mixture of a polyurethane with a copolymer of vinylidene fluoride and hexafluoro-propylene, in particular those prepared as described in the document U.S. Pat. No. 5,349,003. As a variant, they are commercially available in the form of a colloidal dispersion in water, in a ratio of the fluorinated copolymer to the acrylic polymer lying between 70:30 and 75:25, under the trade names KYNAR RC-10.147 and KYNAR RC-10.151 from Atofina.
Examples of grafted silicone polymers are indicated in the document EP-A-1,038,519, which is incorporated here by reference. A preferred example of a grafted silicone polymer is polysilicone-8 (CTFA name) which is a polydimethylsiloxane onto which are grafted, via a linking unit of the thiopropylene type, mixed polymeric moieties of the poly(meth)acrylic acid type and the alkyl poly(meth)acrylate type. A polymer of this type is in particular available under the trade name VS 80 (10% in water) or LO 21 (in powder form) from 3M. This is a copolymer of polydimethylsiloxane with propylthio, methyl acrylate, methyl methacrylate and methacrylic acid groups.
The aforesaid synthetic polymers can be in the form of a latex. As appropriate latexes capable of being used as toning agents, polyester-polyurethane and polyether-polyurethane dispersions, such as those marketed under the names Avalure UR410 and UR460 by Noveon, and under the names Neorez R974, Neorez R981, Neorez R970, as well as acrylic copolymer dispersions such as those marketed under the name Neocryl XK-90 by Avecia can in particular be cited.
Finally, synthetic polymers appropriate as toning polymers can be water-soluble or water-dispersible polymers containing water-soluble or water-dispersible units and containing LSCT units, the said LCST units exhibiting, in particular, a demixing temperature in water of 5 to 40° C. at a concentration of 1% by weight. This type of polymer is more fully described in the document FR-A-2,819,429.
The polymers of natural origin usable as toning agents can be selected from:
   plant proteins and hydrolysates of plant proteins;
   polysaccharides of plant origin, possibly in the form of microgels, such as starch;
   and latexes of plant origin.
As examples of plant proteins and hydrolysates of plant proteins usable as toning agents, proteins and protein hydrolysates from maize, rye, wheat, buckwheat, sesame, spelt, peas, beans, lentils, soya and lupin can be cited.
The polysaccharides usable as toning agents can be selected from polysaccharides of natural origin, capable of forming thermoreversible or crosslinked gels as well as solutions. “Thermoreversible” is understood to mean the fact that the gel state of these polymer solutions is obtained reversibly, once the solution has been cooled below the gelling temperature characteristic of the polysaccharide used. As polysaccharides of natural origin of this type, the carraghenans and quite especially kappa carraghenan and iota carraghenan, agars, gellans, alginates, pectins, chitosans and derivatives thereof, pullulans and derivatives thereof can be cited. The toning polysaccharides can be present in the form of microgels as described in the document FR-A-2,829,025.
The polysaccharides can also be selected from starch and derivatives thereof. The starch can be of any origin: for example rice, maize, potato, cassava, peas, wheat or oats, and it can be natural or possibly modified by a treatment of the crosslinking, acetylation or oxidation type. It can possibly be grafted. As a starch capable of being used as a toning agent, that marketed by Lambert-Rivière under the name Remi Dri can for example be cited.
Another class of toning agents usable according to the invention is constituted of mixed silicates. This expression is understood to mean all silicates of natural or synthetic origin containing several types of cations selected from the alkali metals (for example Na, Li, K) or alkaline earth metals (for example Be, Mg, Ca) and the transition metals. Phyllosilicates, namely silicates having a structure in which the SiO₄ tetrahedra are organized in sheets between which the metal cations are enclosed, are preferably used. A family of silicates particularly preferred as toning agents is that of the laponites. The laponites are silicates of magnesium, lithium and sodium having a layer structure similar to that of the montmorillonites. Laponite is the synthetic form of the natural mineral called “hectorite”. The laponite marketed under the name Laponite XLS or Laponite XLG by Rockwood can for example be used.
Yet another class of toning agents is constituted of microparticles of wax. These are particles having a diameter generally less than 5 μm, or better than 0.5 μm, and essentially constituted of one wax or a mixture of waxes selected for example from Carnauba, Candelilla or Alfa waxes. The melting point of the wax or the mixture of waxes preferably lies between 50° C. and 150° C.
In a further variant, colloidal particles of inorganic fillers can be used as the toning agent. “Colloidal particles”, is understood to mean colloidal particles dispersed in an aqueous, aqueous alcoholic or alcoholic medium, having a number average diameter lying between 0.1 and 100 nm, preferably between 3 and 30 nm. As examples of inorganic fillers, silica, cerium oxide, zirconium oxide, alumina, calcium carbonate, barium sulphate, calcium sulphate, zinc oxide and titanium dioxide can be cited. A particularly preferred inorganic filler is silica. Colloidal particles of silica are in particular available in the form of an aqueous dispersion of colloidal silica from Catalysts & Chemicals under the trade names COSMO S-40 and COSMO S-50. Composite silica-alumina colloidal particles, such as those marketed by Grace under the names Ludox AM, Ludox HSA and Ludox TMA can also be used.
   18) As myorelaxant agents which are agents for smoothing expression wrinkles, the sapogenins (see application EP-A-1,352,643), adenosine (see application FR-0214828), antagonists of the receptors associated with the calcium channels (see application FR-A-2,793,681), and in particular manganese and salts thereof (see application FR-A-2,809,005) and alverine (see application FR-A-2,798,590), agonists of the receptors associated with the chlorine channels, among them glycine (see application EP-A-0704210) and certain extracts of Iris pallida (see application FR-A-2,746,641) can for example be cited.
   19) As sugars, monosaccharides such as D-mannose, L-rhamnose, polysaccharides, C-glycosides and derivatives thereof, such as C-β-D-xylopyranoside-n-propan-2-one, C-β-D-(3,4,5-triacetoxy)xylopyranoside-n-propan-2-one, C-β-D-xylopyranoside-2-hydroxy-propan-2-one and mixtures thereof can be cited.
   IV) Antimicrobial or antifungal active ingredients and preservatives, in particular hexamidine isethionate, undecylenic acid and salts thereof, benzoyl peroxide, benzoic acid and salts thereof, phytic acid and salts thereof, salicylic acid and salts thereof, sorbic acid and salts thereof, levulinic acid, benzyl alcohol, dehydroacetic acid and salts thereof, anisic acid, the acid N-acetyl-L-cysteine, lipoic acid, azelaic acid and salts thereof, arachidonic acid, resorcinol, 2,4,4′-trichloro-2′-hydroxidiphenyl ether, octopirox, octoxy-glycerine, octanoylglycine, capryloyl glycine, farnesol, phytosphingosines, selenium derivatives, zinc pyrithione, chlophenesine, phenoxyethanol, the esters of parahydroxybenzoic acid also called Parabens, formaldehyde liberators such as imidazolidinyl urea or diazolidinyl urea, haloalkynyl carbamates such as 3-iodo-2-propynyl butylcarbamate (IPBC), caprylyl glycol also called 1,2-octanediol, pentylene glycol, N-(3-chloroallyl)hexaminium (or Quaternium-15) chloride, polyhexamethylene biguanide hydrochloride (CTFA name: polyaminopropyl biguanide), alkyltrimethyl-ammonium bromides such as dodecyltrimethylammonium bromide, myristyltrimethylammonium bromide, hexadecyl-trimethylammonium bromide, and mixtures thereof. These preservatives can in particular be present in the aqueous composition.
   V) Anti-inflammatory or soothing active ingredients such as the pentacyclic triterpenes and extracts from plants (e.g. Glycyrrhiza glabra) containing them such as β-glycyrrhetinic acid and salts thereof and/or derivatives thereof (glycyrrhetinic acid mono-glucuronide, stearyl glycyrrhetinate and 3-stearoyloxy glycyrrhetic acid), ursolic acid and salts thereof, oleanolic acid and salts thereof, betulinic acid and salts thereof, extracts of Paeonia suffruticosa, lactiflora, Laminaria saccharina, camomile, Pygeum, Boswellia serrata, Centipeda cunninghami, Helianthus annuus, Linum usitatissimum, Cola nitida, clove, Epilobium Angustifolium or Bacopa monieri, salts of salicylic acid and in particular zinc salicylate, the phycosaccharides from Codif, canola oil, bisabolol, allantoin, Sepivital EPC (phosphate diester of vitamin E and C) from Seppic, omega 3 unsaturated oils such as the oils of muscat rose, blackcurrant and Echium and fish oils, capryloyl glycine, Seppicalm VG (sodium palmitoylproline and nymphea alba) from Seppic, tocotrienols, piperonal, aloe vera and phytosterols.
   VI) Lipolytic or slimming active ingredients, in other words those having a favourable, direct or indirect action on the diminution of adipose tissue, such as:
xanthine derivatives such as caffeine and derivatives thereof, in particular the 1-hydroxy-alkylxanthines described in the document FR-A-2,617,401, caffeine citrate, theophylline and derivatives thereof, theobromine, acefylline, aminophylline, chloroethyl-theophylline, diprofylline, diniprophylline, etami-phylline and derivatives thereof, etofylline, proxy-phylline, or combinations containing xanthine derivatives, such as the combination of caffeine and silanol (methylsilanetriol derivative of caffeine), and for example the product marketed by Exsymol under the name cafeisilane C, or else the compounds of natural origin containing xanthine bases and in particular caffeine, such as extracts of tea, coffee, guarana, maté, cola (Cola Nitida) and in particular the dry extract of guarana fruit (Paulina sorbilis) containing 8 to 10% of caffeine, and ephedrine and derivatives thereof which can in particular be found in the natural state in plants such as Ma Huang (Ephedra plant);
plant extracts and extracts of marine origin, which are either active on the receptors to be inhibited, such as the β-2 blockers and NPY blockers (described in the document EP-A-838217), or inhibit the synthesis of the receptors for LDL or VLDL, or are active in stimulating the β receptors and G proteins, leading to the activation of adenylcyclase. As plant extracts of this type, the following can for example be cited:
   Garcinia Cambogia,
   extracts of Bupleurum chinensis,
   extracts of climbing ivy (Hedera Helix), arnica (Arnica Montana L), rosemary (Rosmarinus officinalis N), marigold (Calendula officinalis), sage (Salvia officinalis L), ginseng (Panax ginseng), St John's wort (Byperycum Perforatum), Butcher's broom (Ruscus aculeatus L), meadowsweet (Filipendula ulmaria L), orthosiphon (Orthosiphon Stamincus Benth), birch (Betula alba), cecropia and argan,
   extracts of ginkgo biloba,
   extracts of horsetail,
   extracts of escin,
   extracts of cang zhu,
   extracts of Chrysanthellum indicum,
   extracts of dioscoreaceae rich in diosgenin or pure diosgenin or hecogenin and derivatives thereof.
   extracts of plants of the genera Armeniacea, Atractylodis Platicodon, Sinommenum, Pharbitidis or Flemingia,
   extracts of Coleus such as C. Forskohlii, C. blumei, C. esquirolii, C. scutellaroids, C. xanthantus and C. Barbatus, such as the extract of Coleus Barbatus root containing 60% of forskolin,
   extracts of black horehound,
   extracts of Guioa, Davallia, Terminalia, Barringtonia, Trema or Antirobia.
As extract of marine origin the following can be cited:
   extracts of algae or phytoplankton, such as rhodysterol or the extract of Laminaria Digitata marketed under the name PHYCOX75 by Secma, the alga skeletonema described in the document FR-A-2,782,921 or the diatoms described in the document FR-A-2,774,292.
      VII) Fillers, and especially those which contribute optical effects, in other words fillers capable of conferring, after application onto the skin, the following optical effects:
matte effect or diminution in shine;
soft focus effect: “Soft focus” is understood to mean the visible diminution of small wrinkles, pores and irregularities in the micro-relief of the skin, a diminution obtained instantly after application of the cosmetic composition onto a skin exhibiting imperfections connected with irregular micro-relief;
homogenization and lightening of the colouring: “Homogeneity of the colouring” is understood to mean the visible diminution of the discolourations and pigmental irregularities of the skin, obtained instantly after application of the cosmetic composition onto a skin exhibiting these imperfections.

The fillers are solid particles, generally white, and insoluble in the medium of the composition.

Among the optical effect fillers usable in the invention, inorganic fillers (amorphous or crystalline titanium dioxide in the form of rutile and/or anatase, zinc oxide, iron oxide, cerium oxide, silica, alumina, boron nitride, talc, sericite, mica, . . . ) coated or uncoated, as well as composite fillers, nacres, clays, starch and derivatives thereof, aqueous dispersions of acrylic styrene, melamine-formaldehyde or urea-formaldehyde resin particles, aqueous dispersions of polytetrafluoroethylene (PTFE), microdispersions of waxes, vinylpyrrolidone/1-triacontene copolymers, silicone waxes and resins, organopolysiloxane particles, expanded vinylidene chloride, acrylonitrile and methacrylate terpolymer microspheres, nylon particles, cellulose microbeads, fibres and hollow hemispherical silicone particles such as those marketed under the names NLK-500 and NLK-503 by Takemoto Oil and Fat can be cited.

Mention may be made of silicon elastomers (organopolysiloxane elastomers), preferably partially or completely crosslinked, in the form of particles such as those sold under the references KSG 15, KSG 16, KSG 17, KSG 18, KSG 26A, KSG 26B, KSG-31″, “KSG-32”, “KSG-33”, “KSG-41”, “KSG-42”, “KSG-43”, “KSG-44” by Shin-Etsu.

VIII) Sunscreens, which can be selected from the UVA and UVB chemical filters or physical filters commonly usable in the cosmetic field.

As UVB filters, the following can for example be cited:

(1) salicylic acid derivatives, in particular homomethyl salicylate and octyl salicylate;
(2) cinnamic acid derivatives, in particular 2-ethylhexyl p-methoxycinnamate, marketed by Givaudan under the name Parsol MCX;
(3) liquid β,β′-diphenylacrylate derivatives, in particular 2-ethylhexyl α-cyano-α, β′ diphenylacrylate or octocrylene, marketed by BASF under the name UVINUL N539;
(4) p-aminobenzoic acid derivatives;
(5) the 4-methyl benzylidene camphor marketed by Merck under the name EUSOLEX 6300;
(6) the 2-phenylbenzimidazole 5-sulphonic acid marketed under the name EUSOLEX 232 by Merck;
(7) 1,3,5-triazine derivatives, in particular:
the 2,4,6-tris[p-(2′-ethylhexyl-1′-oxycarbonyl)-anilino]-1,3,5-triazine marketed by BASF under the name UVINUL T150, and
the dioctyl butamido triazone marketed by Sigma 3V under the name UVASORB HEB;
   (8) and mixtures of these filters.

As UVA filters, the following can for example be cited:

(1) dibenzoylmethane derivatives, in particular the 4-(tert.-butyl) 4′-methoxy dibenzoyl-methane marketed by Givaudan under the name PARSOL 1789;
(2) benzene 1,4 [di(3-methylidenecamphor-10-sulphonic)] acid possibly in partially or totally neutralized form, marketed under the name MEXORYL SX by Chimex.
(3) benzophenone derivatives, for example:
2,4-dihydroxybenzophenone (benzophenone-1);
2,2′,4,4′-tetra-hydroxybenzophenone (benzophenone-2);
2-hydroxy-4-methoxy-benzophenone (benzophenone-3), marketed under the name UVINUL M40 by BASF;
2-hydroxy-4-methoxy-benzophenone-5-sulphonic acid (benzophenone-4) as well as its sulphonate form (benzophenone-5), marketed by BASF under the name UVINUL MS40;
2,2′-dihydroxy-4,4′-dimethoxybenzophenone (benzophenone-6);
5-chloro-2-hydroxybenzophenone (benzophenone-7);
2,2′-dihydroxy-4-methoxy-benzophenone (benzophenone-8);
the disodium salt of 2,2′-dihydroxy-4,4′-dimethoxy-benzophenone-5,5′-disulphonic acid (benzophenone-9);
2-hydroxy-4-methoxy-4′-methyl-benzophenone (benzophenone-10);
benzophenone-11; and
2-hydroxy-4-(octyloxy)benzophenone (benzophenone-12).
   (4) silane derivatives or polyorganosiloxanes with benzophenone group(s);
   (5) anthranilates, in particular the menthyl anthranilate marketed by Haarman & Reiner under the name NEO HELIOPAN MA;
   (6) compounds containing at least two benzoazolyl groups or at least one benzodiazolyl group per molecule, in particular the 1,4-bis-benzimidazolyl-phenylen-3,3′,5,5′-tetrasulphonic acid as well as salts thereof marketed by Haarman & Reimer;
   (7) silicon-containing derivatives of N-substituted benzimidazolyl-benzazoles or benzofuranyl-benzazoles, and in particular:
2-[1-[3-[1,3,3,3-tetramethyl-1-[(trimethylsilyl)oxy]-disiloxanyl]propyl]-1H-benzimidazol-2-yl]-benzoxazole;
2-[1-[3-[1,3,3,3-tetramethyl-1-[(trimethylsilyl)oxy]-disiloxanyl]propyl]-1H-benzimidazol-2-yl]-benzothiazole;
2-[1-(3-trimethylsilanyl-propyl)-1H-benzimidazol-2-yl]-benzoxazole;
6-methoxy-1,1′-bis-(3-trimethylsilanyl-propyl)-1H,1′H-[2,2′]bibenzimidazolyl-benzoxazole;
and 2-[1-(3-trimethylsilanyl-propyl)-1H-benzimid-azol-2-yl]-benzothiazole,
   which are described in the document EP-A-1 028 120;
   (8) triazine derivatives, and in particular the 2,4-bis {[4-(2-ethyl-hexyloxy]-2-hydroxyl-phenyl}-6-(4-methoxy-phenyl)-1,3,5-triazine marketed by Ciba Geigy under the name TINOSORB S, and the 2,2′-methylenebis-[6-(2H benzotriazol-2-yl)-4-(1,1,3,3-tetramethyl-butyl)phenol] marketed by Ciba Geigy under the name TINOSORB M;
   (9) benzotriazole silicones, which are described in particular in the document EP-A-0392 883, in particular the benzotriazole silicone of formula:

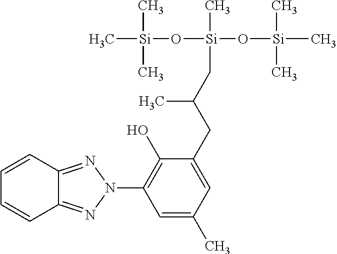

(10) and mixtures thereof.

A mixture of several of these filters can also be used.

IX) Colouring agents for skin and hair, in particular skin colouring agents such as dihydroxyacetone (DHA), natural colorants such as plant extracts such as for example extracts of sorghum, and optical brighteners.

The optical brighteners are a family of fluorescent substances well known to those skilled in the art. Such compounds are described in “Fluorescent Whitening Agent, Encyclopedia of Chemical Technology, Kirk-Othmer”, vol 11, p. 227-241, 4^{th} edition, 1994, Wiley. These are agents for whitening the skin by optical means, constituted of chemical compounds endowed with fluorescent properties, which absorb in the ultraviolet (maximum absorption at a wavelength of less than 400 nm) and reemit energy by fluorescence at a wavelength lying between 380 nm and 830 nm. Emission of energy lying between 400 nm and 480 nm results in emission in the blue of the visible range, which contributes to a visual whitening when this emission takes place on the skin.

They can more particularly be defined as compounds which essentially absorb in the UVA and UVB between 290 and 400 nm and essentially reemit between 400 and 525 nm. Among the optical brighteners, stilbene derivatives, coumarin derivatives, oxazole and benzoxazole derivatives and imidazole derivatives are preferably used. Such compounds are readily available commercially. The optical brighteners preferably used in the product according to the invention are the di-styryl-4,4′ bi-phenyl di-sulphonate marketed by Ciba Geigy under the name Tinopal CBS-X®. The sodium 4,4′-bis[(4,6-dianilino-1,3,5-triazin-2-yl)amino]stilbene-2,2′-disulphonate and the 2,5 thiophen di-yl bis(5 tert.-butyl-1,3 benz-oxazole) marketed by Ciba Geigy under the names Tinopal SOP® and Uvitex OB® can also for example be cited.

X) Concealer active ingredients such as vitamin K1 and derivatives thereof, and coumarins.
XI) Anti-perspirant active ingredients such as aluminium and/or zirconium salts, such as for example perlite, aluminium chlorohydrate, aluminium chloro-hydrex, aluminium chlorohydrex PEG, aluminium chloro-hydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, and alum salts, aluminium sulphate, aluminium zirconium octachlorohydrate, aluminium zirconium pentachloro-hydrate, aluminium zirconium tetrachlorohydrate, aluminium zirconium trichlorohydrate and more particularly the aluminium chlorohydrate marketed by REHEIS under the name REACH 301 or 303 or by GUILINI CHEMIE under the name ALOXICOLL PF 40, and the salt of aluminium and zirconium marketed by REHEIS under the name REACH AZP-908-SUF, complexes of zirconium hydroxychloride and aluminium hydroxychloride with an amino acid such as those described in the document U.S. Pat. No. 3,792,068, commonly known under the name “ZAG complexes”, such as for example aluminium zirconium octachlorohydrex GLY, aluminium zirconium pentachloro-hydrex GLY, aluminium zirconium tetrachlorohydrate GLY and aluminium zirconium trichlorohydrate GLY.
XII) Deodorant active ingredients, such as zinc pyrrolidone carboxylate (commonly called zinc pidolate), zinc sulphate, zinc chloride, zinc lactate, zinc gluconate and zinc phenolsulphonate, 2,4,4′-trichloro-2′-hydroxydiphenyl ether (Triclosan), 2,4-dichloro-2′-hydroxydiphenyl ether, 3′,4′,5′-tri-chlorosalicylanilide, 1-(3′,4′-dichlorophenyl)-3-(4′-chlorophenyl)urea (Triclocarban) or 3,7,11-trimethyl-dodeca-2,5,10-trienol (Farnesol), quaternary ammonium salts such as cetyltrimethylammonium salts, and cetyl-pyridinium salts, chlorhexidine and salts thereof, diglycerol monocaprate, diglycerol monolaurate, glycerol monolaurate and polyhexamethylene biguanide salts.
XIII) Hair treatment active ingredients, such as (1) agents inhibiting hair loss as well as agents stimulating hair growth, such as minoxidil, biotin, aminexil, cysteine, finasteride, 2,4 dipyrimidine N-oxide, panthenol and derivatives, flavanone T, calcium antagonists such as diltiazem, verapamil, alverine, and nifedipine, hormones such as progesterone, FP receptor antagonists such as latanoprost, type 1 15-hydroxy prostaglandin dehydrogenase inhibitors, prostaglandins and derivatives thereof, or more generally any plant extract with type I or II anti-5-alpha reductase activity, or (2) anti-dandruff agents such as zinc pyrithion, 1-hydroxy-2-pyrrolidone derivatives or again selenium sulphides.
XIV) Depilatory agents which are used to inhibit the growth of hair, such as thioglycolic acid and derivatives thereof, dithioglycolic acid and derivatives thereof, the serine proteases described in the document U.S. Pat. No. 6,407,056, caffeic acid, quercetin, propyl gallate, norhydroguaiaretic acid or NDGA, indomethacin, eflornithine hydrochloride; plant extracts as described in the document U.S. Pat. No. 6,171,595, such as extracts of cloves, rosebud, burnet, or gambier, the compounds described in the document U.S. Pat. No. 6,075,052, tetramisole, sodium orthovanadate, levamisole, disodium cromoglycate, vanadium nitrate and gallium nitrate as described in the document U.S. Pat. No. 6,020,006, the compounds described in the documents U.S. Pat. No. 4,885,289, U.S. Pat. No. 4,720,489, U.S. Pat. No. 5,132,293, U.S. Pat. No. 5,096,911, U.S. Pat. No. 5,095,007, U.S. Pat. No. 5,143,925, U.S. Pat. No. 5,328,686, U.S. Pat. No. 5,440,090, U.S. Pat. No. 5,364,885, U.S. Pat. No. 5,411,991, U.S. Pat. No. 5,648,394, U.S. Pat. No. 5,468,476, U.S. Pat. No. 5,475,763, U.S. Pat. No. 5,455,608, U.S. Pat. No. 5,674,477, U.S. Pat. No. 5,728,736 and U.S. Pat. No. 5,652,273, WO-A-94/27586, WO-A-94/27563 and WO-A-98/03149 and extracts of juniper as described in the document U.S. Pat. No. 6,375,948.
XV) Pigments which are used in particular when the product obtained is intended for use as make-up for the skin or, as regards metallic oxide pigments, when the product obtained is intended to be part of a sunscreen product to protect the skin or coloured hair from the sun's rays.

These pigments can be inorganic and/or organic, interference, goniochromatic, fluorescent, white, coloured, nacreous or reflective or in flake form. Pigment should be understood to mean particles insoluble in the physiological medium of the composition.

Among the inorganic pigments, titanium dioxide, possibly surface-treated, zirconium or cerium oxides, as well as zinc oxides, iron oxides (black, yellow or red) or chromium oxides, manganese violet, ultramarine blue, chromium hydrate and ferric blue can be cited. Among the organic pigments, carbon black, organic lake-type pigments of barium, strontium, calcium or aluminium including those which have undergone certification by the Food and Drug Administration (FDA) (for example D&C or FD&C) and those exempt from the FDA certification such as cochineal carmine-based lakes can be cited.

The nacreous pigments or nacres can be selected from nacreous white pigments such as mica coated with titanium or bismuth oxychloride, coloured nacreous pigments such as titanium mica with iron oxides, titanium mica with in particular ferric blue or chrome oxide, titanium mica with an organic pigment of the type previously cited as well as nacreous pigments based on bismuth oxychloride. Among the nacres available commercially, the nacres marketed under the names TIMICA® and FLAMENCO® by Engelhard and the nacres marketed under the name TIMIRON® by Merck can be cited.

Goniochromatic pigments, like pigments with a multilayer interference structure for example of Al/SiO₂/Al/SiO₂/A1 structure marketed by Dupont de Nemours, of Cr/MgF₂/Al/MgF₂/Cr structure marketed under the name CHROMAFLAIR® by Flex, of MoS₂/SiO₂/Al/SiO₂/MoS₂, Fe₂O₃/SiO₂/Al/SiO₂/Fe₂O₃ or Fe₂O₃/SiO₂/Fe₂O₃/SiO₂/Fe₂O₃ structure marketed under the name of SICOPEARL® by BASF or of MoS₂/SiO₂/mica-oxide/SiO₂/MoS₂, Fe₂O₃/SiO₂/mica-oxide/SiO₂/Fe₂O₃, TiO₂/SiO₂/TiO₂ or TiO₂/Al₂O₃/TiO₂ structure marketed under the name XIRONA® by Merck can also be used. Pigments marketed under the name INFINITE COLORS® from Shiseido can also be cited.

Reflective pigments, such as particles of glass substrate coated in silver, in the form of platelets, such as those sold for example under the name MICROGLASS METASHINE REFSX 2025 PS® by Toyal, particles of glass substrate coated in nickel/chromium/molybdenum alloy, such as those sold for example under the names CRYSTAL STAR GF 55®, GF 2525® by Toyal, REFLECKS® brand pigments, marketed by Engelhard, containing a glass substrate coated with brown iron oxide and particles containing a stack of at least two layers of polymers such as those marketed by 3M under the name MIRROR GLITTER can also be used.

As goniochromatic particles with liquid crystals, those sold by Chenix as well as those marketed under the name HELICONE® HC by Wacker can for example be used.

XVI) Colorants which impart a colour to film(s) and/or to the aqueous composition, in particular water-soluble colorants such as copper sulphate, iron sulphate, water-soluble sulphopolyesters, rhodamines, natural colorants such as carotene and beetroot juice, methylene blue, caramel, disodium tartrazine salt and disodium fuchsine salt, and mixtures thereof. Liposoluble colorants can also possibly be used. The colorant or colorants are preferably present in the film or films. The aqueous composition can thus be coloured at the time of use depending on the shade desired at that time.

The colorants and pigments make it possible to adjust the colour to achieve the desired outcome (healthy glow, or concealant effect for example).

XVII) Perfumes which can be of any type, either composite perfumes containing a mixture of odoriferous materials or a single odoriferous material. Thus the kit can include several films, each one containing a different odoriferous material such that several films mixed into the aqueous composition give a particular perfume. Perfumes with myorelaxant properties can also be introduced, giving a relaxant effect on application of the product onto the skin.

The odoriferous materials are compounds commonly used by perfumers and they are in particular described in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials-1991”, Allured Publishing Co. Wheaton, Ill. USA.

They can be natural products (essential oils, absolutes, resinoids, resins or concretes) and/or synthetic products (saturated or unsaturated, aliphatic or cyclic hydrocarbons, alcohols, aldehydes, ketones, ethers, acids, esters, acetals, ketals and nitriles).

XIX) Electrolytes, and in particular salts of mono-, di- or trivalent metals, and more particularly alkaline earth metal salts and in particular salts of barium, calcium and strontium, alkali metal salts and for example salts of sodium and potassium, as well as salts of magnesium, zinc, manganese, iron, copper, aluminium, silicon, selenium, and mixtures thereof.

The ions constituting these salts can for example be selected from carbonates, bicarbonates, sulphates, phosphates, sulphonates, glycerophosphates, borates, bromides, chlorides, nitrates, acetates, hydroxides and persulphates as well as the ions of α-hydroxyacids (citrates, tartrates, lactates, malates) or fruit acids, ions of β-hydroxy-acids (salicylates, 2-hydroxy-alkanoates, n-alkyl salicylates and n alkanoyl-salicylates), or again ions of amino acids (aspartate, arginate, glycocholate, fumarate).

XX) pH adjusters and in particular those commonly used in the cosmetic field to adjust the pH of compositions to the desired value. These can be acids or bases, selected from inorganic bases such as sodium hydroxide, organic bases such as amines (for example triethanolamine), inorganic acids such as hydrochloric acid and organic acids such as citric acid.

The compounds cited above can be present in compositions A and/or B in the quantities usual in the field in question, these quantities depending on the compound used and the desired aim. The compounds can for example be present in a quantity ranging from 0.001 to 30% by weight, and better from 0.01 to 20% by weight relative to the weight of the final composition A or B.

The kit according to the invention can in particular be used to obtain products intended to be applied to the skin, the mucous membranes, integuments, or the hair, in particular as skin care products or as skin make-up products, for removing make-up or cleaning the skin or as hair care products or as sunscreen products. According to one implementation mode, the kit according to the invention contains, in addition to the compositions A and B, at least one additional composition which can for example contain cosmetic active ingredients such as the aforesaid.

In this implementation mode, the final product in emulsion form is preferably prepared by:
introduction of the additional composition containing the active ingredients into composition B, with manual stirring, then
mixing of the above composition with composition A.

The following examples of compositions according to the invention are given for illustration and are not limiting. The quantities are given in % by weight here, unless otherwise stated, and names are stated as the chemical name or CTFA name as appropriate.

### Example 1

### Kit for Skin Care Product

### Composition A

| | |
|---|---|
| Acrylamide/sodium acrylamido-2-methylpropanesulphonate | 7.8% |
| copolymer in 40% inverse emulsion in Polysorbate 80 | |
| and isohexadecane (Simulgel 600 from Seppic) | |
| Methylglucose sesquistearate | 0.8% |
| (Glucate SS from AMERCHOL) | |
| Ethoxylated methylglucoside sesquistearate (20 EO) | 3.2% |
| (Glucamate SSE-20 from AMERCHOL) | |
| Mixture of ethoxylated palm (200 EO) and ethoxylated | 5.6% |
| copra (7 EO) glycerides in aqueous suspension (55/15/30) | |
| (REWODERM LI-S 80 from EVONIK GOLDSCHMIDT) | |
| 2-ethylhexyl glycerol ether (Sensiva SC 50 from Schulke | 0.5% |
| & Mayr) | |
| Glycerine | 15.5% |
| Squalane | 18.5% |
| Dicaprylyl carbonate | 15.5% |
| Shea butter liquid fraction | qsp 100% |
| Beeswax | 4% |
| Tocopherols | 1% |

### Procedure

The dicaprylyl carbonate, glycerine, methylglucose sesquistearate, ethoxylated methylglucoside sesquistearate (20 EO), mixture of ethoxylated palm (200 EO) and ethoxylated copra (70 EO) glycerides and beeswax are heated at 60° C. with high-speed stirring until completely dissolved,
the squalane, rhea butter liquid fraction, 2-ethyl-hexyl glycerol ether and tocopherols are added at 55° C. with high-speed stirring, and the whole is homogenized.
This is allowed to cool to 25° C.,
The acrylamide/acrylamido-2-methylpropanesulphon-ate copolymer is added at 25° C., with high-speed stirring.

### Aqueous Composition B

| | |
|---|---|
| Ethanol | 15% |
| Water | qsp 100% |

During use, the consumer introduces composition B into a pot then slowly adds composition A while stirring with a spatula.

After several seconds of stirring, a smooth and uniform emulsion is formed.

The product thus formed can be stored in the refrigerator (for a period of 1 to 8 weeks after preparation).

### Example 2

### Skin-Care Kit

### Composition A

| | |
|---|---|
| Acrylamide/sodium acrylamido-2-methylpropanesulphonate | 9% |
| copolymer in 40% inverse emulsion in Polysorbate 80 | |
| and isohexadecane (Simulgel 600 from Seppic) | |
| Sorbitan trioleate (NIKKOL SO 30V from NIHON | 1.4% |
| SURFACTANT) | |
| Polyethoxylated glyceryl triisostearate (20 EO) | 5.5% |
| (UNIOX GT 20IS L from NOF) | |
| Polyethylene sorbitan trioleate (20 EO) | 9% |
| (RHEODOL TW - O320 V from Kao) | |
| 2-ethylhexyl glycerol ether (Sensiva SC 50 from Schulke | 0.5% |
| & Mayr) | |
| Dextrin palmitate | 5% |
| (RHEOPEARL KL-2 OR from Chiba Flour) | |
| Glycerine | 15.5% |
| Squalane | 18.5% |
| Dicaprylyl carbonate | 15.5% |
| Shea butter liquid fraction | qsp 100% |
| Beeswax | 3% |
| Tocopherols | 1% |

### Procedure

The dicaprylyl carbonate, glycerine, beeswax, sorbitan trioleate, polyethoxylated glyceryl tri-isostearate (20 EO) and polyethylene sorbitan trioleate (20 EO) are heated at 90° C. with high-speed stirring.
The dextrin palmitate is added and the mixture homogenized until complete solution
This is cooled and the temperature maintained at 55° C. for several minutes to homogenize,
The squalane, shea butter, 2-ethylhexyl glycerol ether and tocopherols are added at 55° C. with high-speed stirring,
This is cooled to 25° C. and
   The acrylamide/sodium acrylamido-2-ethylpropanesulphonate copolymer is added at 25° C. with high-speed stirring.

### Aqueous Composition B

| | |
|---|---|
| 1,3-propanediol | 7% |
| Ethanol | 15% |
| Water | qsp 100% |

During use, the consumer introduces composition B into a pot then adds composition A slowly and while stirring with a spatula.

After several seconds of stirring, a smooth and uniform emulsion is formed.

The product thus formed can be stored in the refrigerator (for a period of 1 to 8 weeks after preparation).

### Example 3

### Kit

### Composition A

| | |
|---|---|
| Sodium acrylamide/acrylamido-2-methylpropanesulphonate | 7.8% |
| copolymer in 40% inverse emulsion in Polysorbate 80 | |
| and isohexadecane (Simulgel 600 from Seppic) | |
| Methylglucose sesquistearate | 0.8% |
| (Glucamate SS from AMERCHOL) | |
| Ethoxylated methylglucoside sesquistearate (20 EO) | 3.2% |
| (Glucamate SSE-20 from AMERCHOL) | |
| Mixture of ethoxylated palm (200 EO) and ethoxylated | 5.6% |
| copra (7 EO) glycerides in aqueous suspension (55/15/30) | |
| (REWODERM LI-S 80 from EVONIK GOLDSCHMIDT) | |
| 2-ethylhexyl glycerol ether (Sensiva SC 50 from Schulke | 0.5% |
| & Mayr) | |
| Hydrogenated C36 diacid/ethylene diamine condensate, | 10% |
| esterified with stearyl alcohol (Uniclear 100 VG from | |
| Arizona Chemicals) | |
| Glycerine | 15.5% |
| Squalane | 18.5% |
| Dicaprylyl carbonate | 15.5% |
| Shea butter liquid fraction | qsp 100% |
| Tocopherols | 1% |

### Procedure

The dicaprylyl carbonate, glycerine, methylglucose sesquistearate, ethoxylated methylglucoside sesquistearate (20 EO) and mixture of ethoxylated palm (200 EO) and ethoxylated copra (70 EO) glycerides are heated at 105-110° C. with high-speed stirring,
The Uniclear is added and the mixture homogenized until complete solution,
This is cooled and the temperature is kept for several minutes at 55° C. to homogenize,
The squalane, shea butter, 2-ethylhexyl glycerol ether and tocopherols are added at 55° C. with high-speed stirring,
This is cooled to 25° C.
The acrylamide/sodium acrylamido-2-methylpropane-sulphonate copolymer is added at 25° C. with high-speed stirring.

### Aqueous Composition B

| | |
|---|---|
| 1,3-propanediol | 7.4% |
| Ethanol | 15% |
| Water | qsp 100% |

During use, the consumer introduces composition B into a pot then adds composition A slowly while stirring with a spatula.

After several seconds of stirring, a smooth and uniform emulsion is formed.

The product thus formed can be stored in the refrigerator (for a period of 1 to 8 weeks after preparation).

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A process for treatment of a keratinous material comprising mixing compositions A and B to form an emulsion and then applying the emulsion to the keratinous material, wherein
composition A comprises at least one oil and at least one gelling agent which is in dispersed form in an organic or aqueous solvent, composition A comprising less than 10% by weight of water;
composition B comprises water and optionally at least one water-soluble organic solvent,
compositions A and B being separate from one another prior to mixing, compositions A and B optionally being separately packed in a single kit prior to mixing.

2. Process according to claim 1, wherein composition A comprises less than 10% by weight of water and has an oil content ranging from 30% to 95% by weight, and composition B is an aqueous composition.

3. Process according to claim 1, wherein compositions A and B are separately packed in a single kit prior to mixing and are mixed together just prior to their application to the keratinous material.

4. Process according to claim 1, wherein composition B exclusively contains water and optionally at least one water-soluble organic solvent.

5. Process according to claim 1, wherein composition B contains at least 70% water.

6. Process according to claim 1, wherein composition A has an oil content of 30% to 95% by weight relative to the total weight of composition A.

7. Process according to claim 1, wherein the gelling agent of composition A is selected from polymers taking the form of an inverse water-in-oil emulsion.

8. Process according to claim 1, wherein the gelling agent of composition A is selected from a) copolymers of an α,β-monoethylenically unsaturated carboxylic acid, b) copolymers based on 2-acrylamido-2-methylpropane sulphonic acid, and mixtures thereof.

9. Process according to claim 1, wherein composition A comprises at least one gelling agent selected from copolymers of 2-acrylamido-2-methylpropane sulphonic acid.

10. Process according to claim 1, wherein the gelling agent is present at a dry matter content of 0.1% to 25% by weight relative to the total weight of the composition A.

11. Process according to claim 1, wherein composition A further comprises at least one nonionic surfactant.

12. Process according to claim 1, wherein composition A further comprises at least one nonionic surfactant selected from polyol esters of fatty acids with a saturated or unsaturated chain containing from 8 to 24 carbon atoms and alkoxylated derivative(s) thereof, polyethylene glycol esters of C₈-C₂₄ fatty acids and alkoxylated derivatives thereof, sorbitol esters of C₈-C₂₄ fatty acids and alkoxylated derivatives thereof, sugar (sucrose, glucose, alkylglucose) esters of C₈-C₂₄ fatty acids and alkoxylated derivatives thereof, ethers of fatty alcohols, ethers of sugar and C₈-C₂₄ fatty alcohols, and mixtures thereof.

13. Process according to claim 1, wherein composition A further comprises at least one lipophilic structuring agent selected from a) polyamide polymers, b) olefin copolymers, c) semi-crystalline polymers, d) dextrin esters of fatty acids, e) pyrogenic silicas, f) organo-philic clays, and mixtures thereof.

14. Process according to claim 1, wherein composition A further comprises at least one lipophilic structuring agent selected from dextrin esters of fatty acid(s).

15. Process according to claim 13, wherein the lipophilic structuring agent represents from 0.5% to 20% by weight relative to the total weight of composition A.

16. Process according to claim 1, wherein composition A comprises less than 5% by weight of water.

17. Process according to claim 1, wherein composition A represents from 2 to 70% by weight relative to the total weight of compositions A and B.

18. Kit for formulation of a cosmetic product in emulsion form comprising:
a composition A comprising at least one oil and at least one gelling agent which is in dispersed form in an organic or aqueous solvent, composition A comprising less than 10% by weight of water and having an oil content ranging from 30% to 95% by weight, and
an aqueous composition B exclusively containing water and optionally at least one water-soluble organic solvent,
compositions A and B being packed separately in said kit.
